(19) United States
(12) Patent Application Publication
(10) Pub. No.: US 2025/0016021 A1
(43) Pub. Date: Jan. 9, 2025

(54) METHOD OF CONTROLLING TEMPERATURE ADJUSTING DEVICE AND SERVER DEVICE FOR PERFORMING THE METHOD

(71) Applicant: Samsung Electronics Co., Ltd., Suwon-si (KR)

(72) Inventors: Juhyeon PAENG, Suwon-si (KR); Byeongcheol YOON, Suwon-si (KR); Younghak KONG, Suwon-si (KR); Hyelim PARK, Suwon-si (KR); Soohyun SUNG, Suwon-si (KR); Yesol LEE, Suwon-si (KR); Myoungsoo JANG, Suwon-si (KR); Chihong CHO, Suwon-si (KR)

(21) Appl. No.: 18/763,415

(22) Filed: Jul. 3, 2024

Related U.S. Application Data
(63) Continuation of application No. PCT/KR2024/008185, filed on Jun. 14, 2024.

(30) Foreign Application Priority Data
Jul. 4, 2023 (KR) 10-2023-0086776
Sep. 7, 2023 (KR) 10-2023-0119277

(51) Int. Cl. H04L 12/28; A61B 5/00; A61B 5/01; A61B 5/0205; A61B 5/024; A61B 5/08

(52) U.S. Cl. CPC H04L 12/282; A61B 5/02055; A61B 5/4812

(57) ABSTRACT

A method of controlling a temperature adjusting device and a server device for performing the method are provided. The method includes obtaining user sleep information comprising at least one of a body temperature value, a heart rate value, or a respiration value of a user, determining a current sleep stage of the user based on the user sleep information, determining whether the body temperature value exceeds a body temperature range corresponding to the current sleep stage, and controlling at least one temperature adjusting device based on a determination that the body temperature value exceeds the body temperature range.

### CROSS-REFERENCE TO RELATED APPLICATION(S)

This application is a continuation application, claiming priority under § 365(c), of an International application No. PCT/KR2024/008185, filed on Jun. 14, 2024, which is based on and claims the benefit of a Korean patent application number 10-2023-0086776, filed on Jul. 4, 2023, in the Korean Intellectual Property Office, and of a Korean patent application number 10-2023-0119277, filed on Sep. 7, 2023, in the Korean Intellectual Property Office, the disclosure of each of which is incorporated by reference herein in its entirety.

### BACKGROUND

### 1. Field

The disclosure relates to a method of controlling a temperature adjusting device, and a server device and control system for performing the method. More particularly, the disclosure relates to a method of adjusting an indoor temperature value by using a correlation between a sleep stage and a body temperature value or between a sleep stage and an indoor temperature value and an electronic device for performing the method.

### 2. Description of Related Art

Recently, technologies for accurately measuring sleep stages have been actively studied. These technologies help to identify and understand sleep states of users by analyzing human brain waves, electrocardiogram, and eye movements, and are being studied to help users experience a more comfortable and high-quality sleep.

Recently, various technologies have been developed to improve the sleep experience of users by using these research results. Applications and devices for monitoring and analyzing sleep stages in real time have been developed to provide accurate feedback to users to help them build personalized sleep schedules and environments. In addition, technologies for optimizing sleep environments of users have been continuously studied.

The above information is presented as background information only to assist with an understanding of the disclosure. No determination has been made, and no assertion is made, as to whether any of the above might be applicable as prior art with regard to the disclosure.

### SUMMARY

Aspects of the disclosure are to address at least the above-mentioned problems and/or disadvantages and to provide at least the advantages described below. Accordingly, an aspect of the disclosure is to provide a method of controlling a temperature adjusting device, and a server device and control system for performing the method.

Additional aspects will be set forth in part in the description which follows and, in part, will be apparent from the description, or may be learned by practice of the presented embodiments.

In accordance with an aspect of the disclosure, a method of controlling a temperature adjusting device is provided. The method includes obtaining user sleep information including at least one of a body temperature value, a heart rate value, or a respiration value of a user, determining a current sleep stage of the user based on the user sleep information, determining whether the body temperature value exceeds a body temperature range corresponding to the current sleep stage, and controlling at least one temperature adjusting device based on a determination that the body temperature value exceeds the body temperature range.

In accordance with another aspect of the disclosure, a server device for controlling a temperature adjusting device is provided. The server device includes memory storing one or more computer programs, one or more processors communicatively coupled to the memory, wherein the one or more computer programs include computer-executable instructions that, when executed by the one or more processors individually or collectively, cause the server device to obtain user sleep information including at least one of a body temperature value, a heart rate value, or a respiration value of a user, determine a current sleep stage of the user based on the user sleep information, determine whether the body temperature value exceeds a body temperature range corresponding to the current sleep stage, and control at least one temperature adjusting device based on a determination that the body temperature exceeds the body temperature range.

In accordance with another aspect of the disclosure, one or more non-transitory computer-readable storage media storing computer-executable instructions that, when executed by one or more processors of a temperature adjusting device individually or collectively, cause the temperature adjusting device to perform operations are provided. The operations include obtaining user sleep information comprising at least one of a body temperature value, a heart rate value, or a respiration value of a user, determining a current sleep stage of the user based on the user sleep information, determining whether the body temperature value exceeds a body temperature range corresponding to the current sleep stage, and controlling at least one temperature adjusting device based on a determination that the body temperature value exceeds the body temperature range.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a conceptual view illustrating a method of controlling a temperature adjusting device, according to an embodiment of the disclosure;

FIGS. 2A and 2B are block diagrams illustrating a system for controlling a temperature adjusting device, according to various embodiments of the disclosure;

FIGS. 3A and 3B are conceptual diagrams illustrating a sleep stage according to sleeping hours, according to various embodiments of the disclosure;

FIG. 4 is a conceptual diagram illustrating an operation of building a user sleep information database (DB) by using an artificial intelligence (AI) model, according to an embodiment of the disclosure;

FIG. 5 is a flowchart illustrating a method of controlling a temperature adjusting device, according to an embodiment of the disclosure;

FIG. 6 is a flowchart illustrating a method of controlling a temperature adjusting device based on external environment information, according to an embodiment of the disclosure;

FIG. 7 is a flowchart illustrating a method of controlling a temperature adjusting device based on an external illuminance value, according to an embodiment of the disclosure;

FIG. 8 is a flowchart illustrating a method of controlling a temperature adjusting device based on an external noise value, according to an embodiment of the disclosure;

FIG. 9 is a flowchart illustrating a method of controlling a temperature adjusting device by comparing an indoor temperature value with an external temperature value, according to an embodiment of the disclosure;

FIG. 10 is a flowchart illustrating a method of controlling a temperature adjusting device based on a noise sensitivity, an illuminance sensitivity, and a temperature sensitivity of a user, according to an embodiment of the disclosure;

FIG. 11 is a flowchart illustrating a method of controlling a temperature adjusting device by comparing an indoor temperature value, a first external temperature value, and a second external temperature value, according to an embodiment of the disclosure;

FIG. 12 is a flowchart illustrating a method of controlling a temperature adjusting device, according to an embodiment of the disclosure;

FIG. 13 is a flowchart illustrating a method of controlling a temperature adjusting device by comparing an indoor temperature value, a first external temperature value, and a second external temperature value, according to an embodiment of the disclosure;

FIG. 14 is a block diagram illustrating elements of a temperature adjusting device, according to an embodiment of the disclosure;

FIG. 15A is a block diagram illustrating elements of an air conditioner, according to an embodiment of the disclosure;

FIG. 15B is a block diagram illustrating elements of an automatic door opening/closing device, according to an embodiment of the disclosure;

FIG. 16 is a block diagram illustrating elements of a wearable device, according to an embodiment of the disclosure; and

FIG. 17 is a block diagram illustrating elements of a server device, according to an embodiment of the disclosure.

Throughout the drawings, it should be noted that like reference numbers are used to depict the same or similar elements, features, and structures.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

When a portion “includes” an element, another element may be further included, rather than excluding the existence of the other element, unless otherwise described. In addition, the term “ . . . unit” or “ . . . module” refers to a unit that performs at least one function or operation, and the unit may be implemented as hardware or software or as a combination of hardware and software.

The expression “configured (or set) to” used in the disclosure may be replaced with, for example, “suitable for,” “having the capacity to,” “designed to,” “adapted to,” “made to,” or “capable of” according to a situation. The term “configured (or set) to” does not always mean only “specifically designed to” by hardware. Alternatively, in some situations, the expression “system configured to” may mean that the system is “capable of” operating together with another device or component. For example, “a processor configured (or set) to perform A, B, and C” may be a dedicated processor (e.g., an embedded processor) for performing a corresponding operation or a generic-purpose processor (e.g., a central processing unit (CPU) or an application processor) that may perform a corresponding operation by executing at least one software program stored in memory.

In addition, in the specification, it will be understood that when elements are “connected” or “coupled” to each other, the elements may be directly connected or coupled to each other, but may alternatively be connected or coupled to each other with an intervening element therebetween, unless specified otherwise.

In the disclosure, functions related to “artificial intelligence (AI)” are performed through a processor and memory. The processor may include one or more processors. In this case, the one or more processors may include a general-purpose processor, such as a central processing unit (CPU), an application processor (AP), or a digital signal processor (DSP), a dedicated graphics processor, such as a graphics processing unit (GPU) or a vision processing unit (VPU), or an AI processor, such as a neural processing unit (NPU). The one or more processors control to process input data according to a predefined operation rule or an AI model stored in the memory. Alternatively, when the one or more processors are AI processors, the AI processors may be designed in a hardware structure specialized in dealing with a specific AI model.

The predefined operation rule or the AI model may be made by training. Specifically, the predefined operation rule or the AI model being made by training refers to the predefined operation rule or the AI model established to perform a desired feature (or a purpose) as a basic artificial intelligence model is trained using a plurality of pieces of training data according to a learning algorithm. Such training may be performed by a device itself in which AI is performed according to the disclosure, or by a separate server and/or system. Examples of the learning algorithm include, but are not limited to, supervised learning, unsupervised learning, semi-supervised learning, and reinforcement learning.

In the disclosure, the term “AI model” may refer to a model for analyzing a linear or non-linear correlation between a plurality of operands (also referred to as variables or parameters). For example, an AI model may include at least one of, but not limited to, linear regression, polynomial regression, logistic regression, decision tree, support vector machine (SVM), or linear correlation neural network models. In an embodiment of the disclosure, an AI model may infer another type of variable by using one type of variable as an input. In an embodiment of the disclosure, an AI model may infer a correlation coefficient between variables by using different types of variables as an input. Examples of a correlation coefficient may include, but are not limited to, a Pearson correlation coefficient, a Spearman correlation coefficient, Kendall's Tau, and a point-biserial correlation coefficient.

In an embodiment of the disclosure, an “AI model” may include a neural network model. The neural network model may include a plurality of neural network layers. The plurality of neural network layers have a plurality of weight values, and a neural network operation is performed through an operation between an operation result of a previous layer and the plurality of weight values. The plurality of weight values of the neural network layers may be optimized by a result of training the AI model. For example, the plurality of weight values may be updated to reduce or optimize a loss value or a cost value obtained by the AI model during a training procedure. An artificial neural network may include a deep neural network (DNN), for example, but not limited to, a convolutional neural network (CNN), a recurrent neural network (RNN), a restricted Boltzmann machine (RBM), a deep belief network (DBN), a bidirectional recurrent deep neural network (BRDNN), or a deep Q-network.

An embodiment of the disclosure will now be described more fully with reference to the accompanying drawings for one of ordinary skill in the art to be able to perform the embodiment of the disclosure without any difficulty. However, the disclosure may be embodied in many different forms and is not limited to the embodiments of the disclosure set forth herein.

Hereinafter, embodiments of the disclosure will be described with reference to the drawings.

It should be appreciated that the blocks in each flowchart and combinations of the flowcharts may be performed by one or more computer programs which include computer-executable instructions. The entirety of the one or more computer programs may be stored in a single memory device or the one or more computer programs may be divided with different portions stored in different multiple memory devices.

Any of the functions or operations described herein can be processed by one processor or a combination of processors. The one processor or the combination of processors is circuitry performing processing and includes circuitry like an application processor (AP, e.g., a central processing unit (CPU)), a communication processor (CP, e.g., a modem), a graphical processing unit (GPU), a neural processing unit (NPU) (e.g., an artificial intelligence (AI) chip), a wireless-fidelity (Wi-Fi) chip, a Bluetooth™ chip, a global positioning system (GPS) chip, a near field communication (NFC) chip, connectivity chips, a sensor controller, a touch controller, a finger-print sensor controller, a display drive integrated circuit (IC), an audio CODEC chip, a universal serial bus (USB) controller, a camera controller, an image processing IC, a microprocessor unit (MPU), a system on chip (SoC), an IC, or the like.

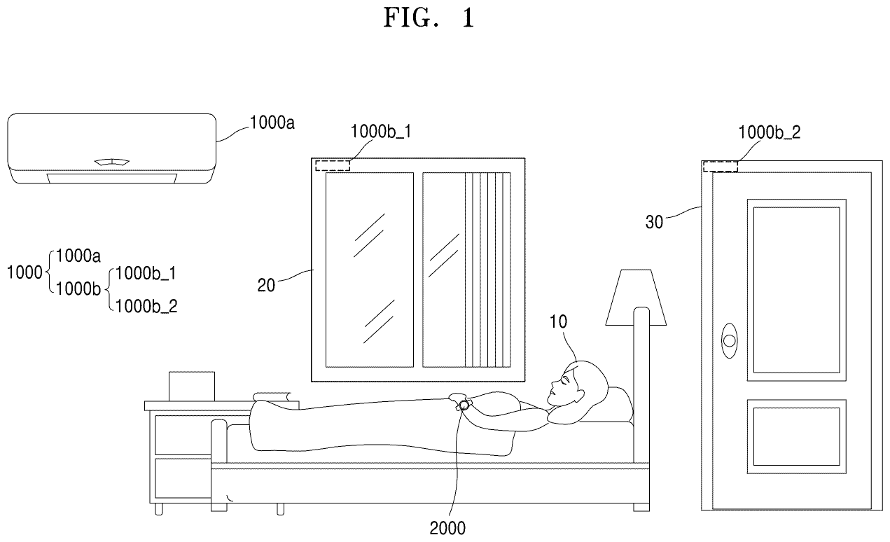

FIG. 1 is a conceptual view illustrating a method of controlling a temperature adjusting device according to an embodiment of the disclosure.

Referring to FIG. 1, when a user 10 sleeps, a sleep state of the user 10 may change stepwise. For example, a sleep state of the user 10 may be expressed as a non-sleep stage and at least one sleep stage. The at least one sleep stage may continuously occur within a sleep cycle and may periodically repeat. The sleep stage may be divided into a rapid eye movement (REM) sleep stage and a non-REM sleep stage. The REM sleep stage may indicate a state where the eyes move quickly and brain wave activity is active. The non-REM sleep stage is a sleep stage other than the REM sleep stage and may be divided into, for example, a sleep stage N1, a sleep stage N2, and a sleep stage N3. The sleep stage N1 is a transitional sleep state and may indicate an initial sleep stage entering from the non-sleep stage to the sleep stage. The sleep stage N2 may indicate a deeper sleep stage than the sleep stage N1. The sleep stage N3 is a deeper sleep stage than the sleep stage N2 and may indicate a deepest sleep stage where delta brain wave patterns appear. Each user 10 may have a different general (or typical) sleep pattern.

As a body temperature of the user 10 changes, a sleep stage of the user 10 may change. An indoor temperature value may be adjusted based on a correlation between a body temperature value of the user 10 and a sleep stage of the user 10. For example, an indoor temperature value may be adjusted by using at least one temperature adjusting device 1000.

In an embodiment of the disclosure, the at least one temperature adjusting device 1000 may be located in an indoor space. The at least one temperature adjusting device 1000 may adjust a temperature of the indoor space (also referred to as an indoor temperature value). In an embodiment of the disclosure, the at least one temperature adjusting device 1000 may perform a cooling or heating function. In an embodiment of the disclosure, the at least one temperature adjusting device 1000 may adjust a temperature of the indoor space by using an outdoor temperature (also referred to as an external temperature value). For example, the at least one temperature adjusting device 1000 may include at least one of an air conditioner 1000a or an automatic door opening/closing device 1000b. However, the disclosure is not limited thereto, and the at least one temperature adjusting device 1000 may include any electronic device for performing a function of adjusting an indoor temperature. For example, the at least one temperature adjusting device 1000 may include an indoor ventilation device.

In an embodiment of the disclosure, the air conditioner 1000a may be a device for appropriately adjusting at least one of a temperature, a humidity, a cleanliness level, or an airflow of indoor air. The air conditioner 1000a may include a remote control device (hereinafter, referred to as a remote controller) for controlling the air conditioner 1000a. In addition, the air conditioner 1000a may obtain indoor environment information through at least one sensor. For example, the air conditioner 1000a may include a temperature sensor, a humidity sensor, and a dust sensor. The air conditioner 1000a may measure a current indoor temperature through the temperature sensor. The air conditioner 1000a may measure a current indoor humidity through the humidity sensor. The air conditioner 1000a may measure a current indoor dust value through the dust sensor. The air conditioner 1000a may receive a control signal from a server device (not shown). The air conditioner 1000a may adjust at least one of a temperature, a humidity, a cleanliness level, or an airflow of indoor air based on the control signal.

In an embodiment of the disclosure, the automatic door opening/closing device 1000b may open and close a window 20 or a room door 30 located in the indoor space. The automatic door opening/closing device 1000b may receive a control signal from the server device (not shown). The automatic door opening/closing device 1000b may open or close the window 20 or the room door 30 based on the control signal. For example, the automatic door opening/closing device 1000b may include at least one of an automatic window opening/closing device 1000b_1 or an automatic room door opening/closing device 1000b_2. The automatic window opening/closing device 1000b_1 may open or close the window 20. The automatic window opening/closing device 1000b_1 may open or close the room door 30. According to an embodiment of the disclosure, as the window or the room door 30 is opened or closed by the automatic door opening/closing device 1000b, indoor air and outdoor air may interact with each other. Accordingly, at least one of a temperature, a humidity, a cleanliness level, or an airflow of the indoor air may be appropriately adjusted.

The user 10 may sleep while wearing a wearable device 2000. Although the wearable device 2000 is a smart watch in FIG. 1, the disclosure is not limited thereto, and the wearable device 2000 may be any electronic device that is worn or attached to a user's body and collects various biometric and activity information. Examples of the wearable device 2000 may include an activity tracker, a smart ring, smart glasses, smart clothing, and a smart headband.

The wearable device 2000 may obtain user sleep information of the user 10 by using a sensor (e.g., a body temperature detection sensor, a respiration detection sensor, or a heart rate detection sensor). For example, the user sleep information may include at least one of, but not limited to, a body temperature value, a respiration value, or a heart rate value of the user 10. For example, the user sleep information may include an arm movement value and an electromyogram value of the user 10. For example, the wearable device 2000 may obtain a body temperature value by using the body temperature detection sensor. The wearable device 2000 may obtain a respiration value by using the respiration detection sensor. The wearable device 2000 may obtain a heart rate value by using the heart rate detection sensor.

The server device (not shown) may obtain the user sleep information from the wearable device 2000. The server device (not shown) may determine a current sleep stage of the user 10 based on the user sleep information. The server device (not shown) may determine whether a body temperature value exceeds a body temperature range corresponding to the current sleep stage. The server device (not shown) may control the temperature adjusting device 1000, based on the determination that the body temperature value exceeds the body temperature range. In an embodiment of the disclosure, the server device (not shown) may control the air conditioner 1000a to operate at a specific set temperature, the window 20 to be opened and closed by using the automatic window opening/closing device 1000b_1, or the room door 30 to be opened and closed by using the automatic room door opening/closing device 1000b_2. According to an embodiment of the disclosure, a body temperature of the user 10 may be maintained at a body temperature corresponding to a high-quality sleep pattern by adjusting an indoor temperature to appropriately maintain or change a sleep stage of the user 10.

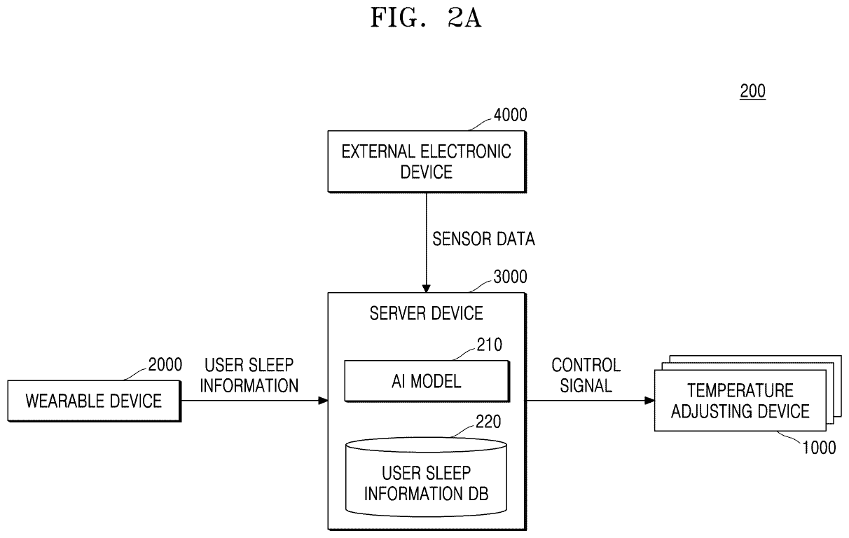

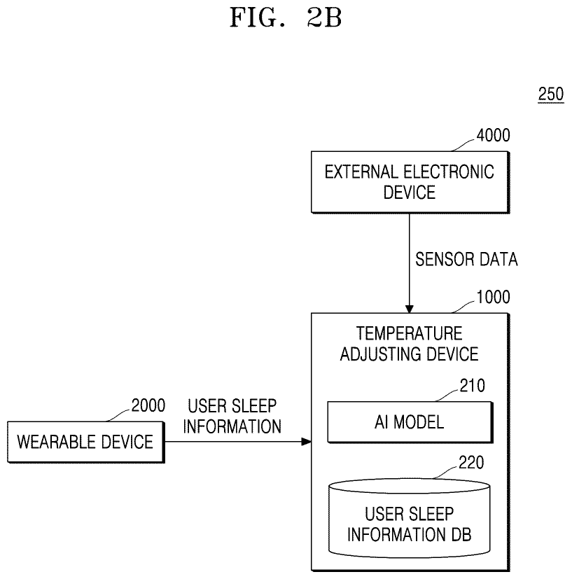

FIGS. 2A and 2B are block diagrams illustrating a system for controlling a temperature adjusting device (hereinafter, referred to as a control system), according to various embodiments of the disclosure. Configurations, operations, and functions of the temperature adjusting device 1000 and the wearable device 2000 may correspond to configurations, operations, and functions of the temperature adjusting device 1000 and the wearable device 2000 of FIG. 1. For convenience of explanation, the same description as that made with reference to FIG. 1 will be omitted.

Referring to FIG. 2A, a control system 200 may include at least one temperature adjusting device 1000, the wearable device 2000, and a server device 3000. However, not all of the illustrated elements are essential elements. The control system 200 may include more or fewer elements than those illustrated in FIG. 2A. For example, the control system 200 may further include an external electronic device 4000. The external electronic device 4000 may transmit sensor data obtained by sensing values corresponding to the outside of an indoor space to the server device 3000. The server device 3000 may obtain external environment information based on the sensor data. For example, the external environment information may include at least one of an external noise value, illuminance value, temperature value, humidity value, or air quality value. For example, the control system 200 may further include a display device (e.g., a mobile terminal) connected to the server device 3000. The display device (not shown) may be a device that executes a certain application provided by the server device 3000 and displays information provided by the server device 3000 through an execution window of the certain application. Hereinafter, each element of the control system according to an embodiment of the disclosure will be described.

In an embodiment of the disclosure, the at least one temperature adjusting device 1000 may include a communication unit (or referred to as a communication interface) for performing communication with an external device. For example, the at least one temperature adjusting device 1000 may communicate with the server device 3000 through the communication interface. The communication unit may include a short-range communication unit and a mobile communication unit. Examples of the short-range communication unit (short-range wireless communication interface) may include, but are not limited to, a Bluetooth communication unit, a Bluetooth low energy (BLE) communication unit, a near-field communication interface, a wireless local area network (WLAN) (Wi-Fi) communication unit, a Zigbee communication unit, an infrared data association (IrDA) communication unit, a Wi-Fi direct (WFD) communication unit, an ultra-wideband (UWB) communication unit, and an Ant+ communication unit. The at least one temperature adjusting device 1000 may receive a control signal from the server device 3000 through the communication unit.

In an embodiment of the disclosure, the at least one temperature adjusting device 1000 may include an air conditioner. The temperature adjusting device 1000 may receive a control signal related to set temperature adjustment. The temperature adjusting device 1000 may receive, but is not limited to, a request to maintain a set temperature, a request to lower a set temperature, a request to increase a set temperature, a request to reduce a wind speed, a request to increase a wind speed, and a request to maintain a wind speed. In an embodiment of the disclosure, the temperature adjusting device 1000 may adjust a set temperature when a control signal related to set temperature adjustment is received from the server device 3000. The temperature adjusting device 1000 may adjust a wind intensity when a request related to wind speed adjustment is received from the server device 3000.

In an embodiment of the disclosure, the at least one temperature adjusting device 1000 may include an automatic door opening/closing device. The at least one temperature adjusting device 1000 may receive a control signal related to opening/closing of a room door or window. The at least one temperature adjusting device 1000 may receive, but is not limited to, room door opening, door closing, window opening, and window closing. In an embodiment of the disclosure, the at least one temperature adjusting device 1000 may open and close a room door or a window when a control signal related to opening/closing of a room door or window is received from the server device 3000.

In an embodiment of the disclosure, the wearable device 2000 may include at least one sensor for obtaining user sleep information and a communication unit for performing communication with an external device. For example, the wearable device 2000 may obtain user sleep information including at least one of a body temperature value, a heart rate value, or a respiration value of a user by using the at least one sensor. For example, the wearable device 2000 may communicate with the server device 3000 through the communication interface. The communication unit may include a short-range communication unit and a mobile communication unit. Examples of the short-range communication unit (short-range wireless communication interface) may include, but are not limited to, a Bluetooth communication unit, a Bluetooth low energy (BLE) communication unit, a near-field communication interface, a wireless local area network (WLAN) (Wi-Fi) communication unit, a Zigbee communication unit, an infrared data association (IrDA) communication unit, a Wi-Fi direct (WFD) communication unit, an ultra-wideband (UWB) communication unit, and an Ant+ communication unit. The wearable device 2000 may transmit the user sleep information (or sensor data corresponding to the user sleep information) to the server device 3000 through the communication unit.

In an embodiment of the disclosure, the server device 3000 may determine a current sleep stage of the user based on the user sleep information. For example, the server device 3000 may determine a current sleep stage of the user by using a correlation between user sleep information and a sleep stage of a user.

In an embodiment of the disclosure, the server device 3000 may include an artificial intelligence (AI) model 210 and a user sleep information database (hereinafter, referred to as DB). In an embodiment of the disclosure, the server device 3000 may be trained about a correlation between a temperature value of a user and a sleep stage of the user by using the AI model 210. However, the disclosure is not limited thereto, and the server device 3000 may be trained about, by using the AI model 210, a correlation between a body temperature value of a user (or sleep quality of the user according to a body temperature of the user) and a sleep stage of the user, a correlation between an indoor temperature value and a sleep stage of a user, a correlation between an indoor humidity value and a sleep stage of a user, a correlation between a body temperature value of a user and an indoor temperature value, a correlation between a noise value around a user (or sleep quality of the user according to a noise value) and a sleep stage of the user, a correlation between an illuminance value around a user (or sleep quality of the user according to an illuminance value) and a sleep stage of the user, or a correlation between at least one of a noise value, an illuminance value, or a body temperature value and a sleep stage of a user.

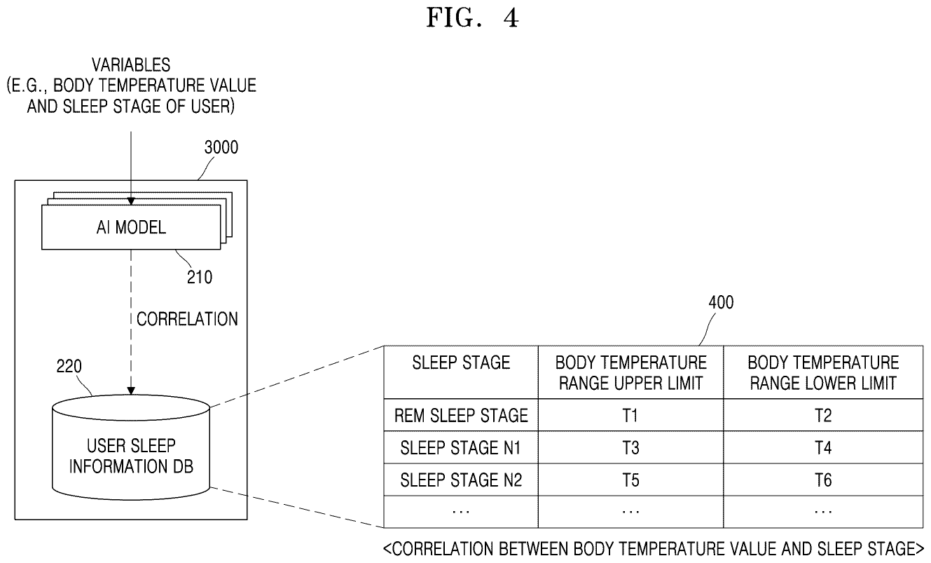

In an embodiment of the disclosure, the server device 3000 may store the learned correlation or a mapping table corresponding to the learned correlation in the user sleep information DB 220. The user sleep information DB 220 will be described below with reference to FIG. 4.

In an embodiment of the disclosure, the server device 3000 may generate a control signal for controlling the temperature adjusting device based on the user sleep information and the sensor data. The server device 3000 may determine whether there is a need to increase or lower a body temperature value of the user, by using a correlation between (i) values corresponding to the user sleep information and the sensor data and (ii) and a sleep stage of the user, previously stored in the user sleep information DB 220. The server device 3000 may adjust an indoor temperature by controlling the temperature adjusting device 1000, based on the determination that there is a need to increase or lower a body temperature value of the user.

In an embodiment of the disclosure, the server device 3000 may adjust an indoor temperature by controlling the temperature adjusting device 1000 so that a sleep stage gradually changes according to a wake-up time preset by the user. For example, the server device 3000 may control the temperature adjusting device 1000 so that, based on a correlation between a specific variable (e.g., a body temperature value or an indoor temperature value) and a sleep stage of the user, the specific variable reaches a threshold value corresponding to a sleep stage change. According to an embodiment of the disclosure, because a sleep stage of the user is changed without significant stimulation, the user may experience a comfortable sleep.

Referring to FIG. 2B, according to an embodiment of the disclosure, unlike the control system 200, the server device 3000 may be omitted from a control system 250. For example, at least some functions of the server device 3000 may be performed by the temperature adjusting device 1000. For example, the temperature adjusting device 1000 may include the AI model 210 and the user sleep information DB 220. The temperature adjusting device 1000 may receive user sleep information from the wearable device 2000. The temperature adjusting device 1000 may receive sensor data from the external electronic device 4000. The temperature adjusting device 1000 may adjust a set temperature or may open and close a window or a room door based on the user sleep information and the sensor data.

In an embodiment of the disclosure, unlike in FIGS. 2A and 2B, the wearable device 2000 may be omitted from the control systems 200 and 250. At least some functions of the wearable device 2000 may be performed by the temperature adjusting device 1000. For example, the temperature adjusting device 1000 may include at least one sensor (or referred to as a sensing unit). The temperature adjusting device 1000 may obtain user sleep information (e.g., a respiration value and a body temperature information) by using the at least one sensor. For example, the temperature adjusting device 1000 may obtain a breathing sound (or referred to as a respiration value) and/or a body temperature (or referred to as a body temperature value) of a user, by using the at least one sensor. In an embodiment of the disclosure, like the wearable device 2000 of FIG. 2A, the temperature adjusting device 1000 may transmit user sleep information to the server device 3000. In an embodiment of the disclosure, like the temperature adjusting device 1000 of FIG. 2B, the temperature adjusting device 1000 may perform at least some functions of the server device 3000 of FIG. 2A. For example, the temperature adjusting device 1000 may determine a current sleep stage of the user based on the user sleep information. For example, the temperature adjusting device 1000 may adjust a set temperature or may open and close a window or a room door based on the user sleep information and sensor data.

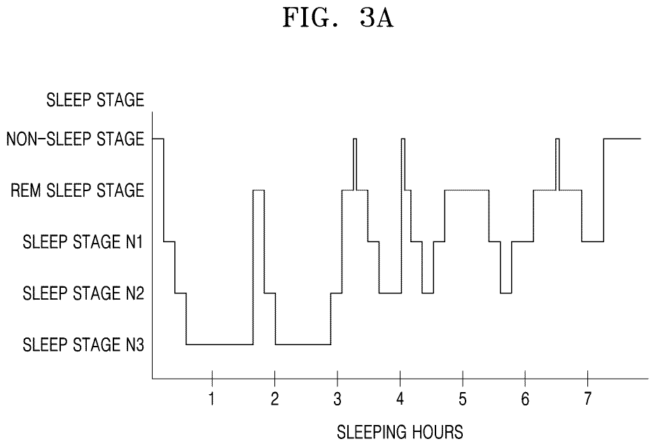

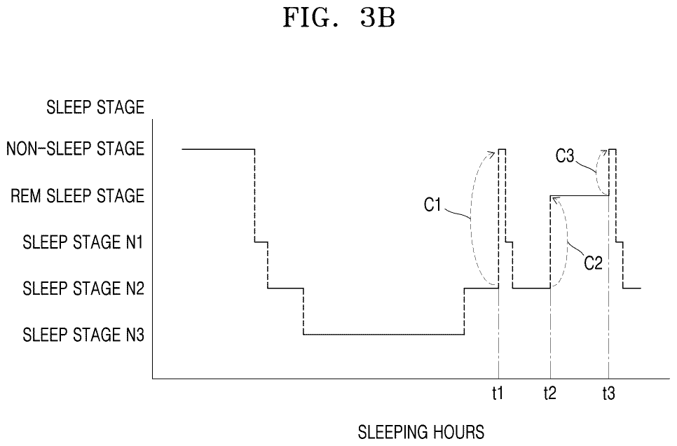

FIGS. 3A and 3B are conceptual diagrams illustrating a sleep stage according to sleeping hours, according to various embodiments of the disclosure. For convenience of explanation, the same description as that made with reference to FIGS. 1 and 2B will be omitted.

Referring to FIG. 3A, a sleep pattern of a user may be defined as a relationship between sleeping hours and a sleep stage. First, when the user in a non-sleep stage falls asleep, a sleep state of the user may change from the non-sleep stage to a non-REM sleep stage. First, the sleep state of the user may correspond to a sleep stage N1 in the non-REM sleep stage. Next, the sleep stage of the user may change from the sleep stage N1 to a sleep stage N2 in the non-REM sleep stage. Next, the sleep state of the user may change from the sleep stage N2 to a sleep stage N3 that is a deepest sleep stage in the non-REM sleep stage. As sleeping hours increase, the sleep state of the user may change from the non-REM sleep stage to a REM sleep stage. The sleep pattern of the user may repeat from the non-REM sleep stage to the REM sleep stage. The sleep pattern of the user may include a plurality of sleep cycles, and one sleep cycle may indicate sleeping hours from the non-REM sleep stage to the REM sleep stage. A sleep pattern may vary according to a user, and a sleep pattern expressed as high-quality sleep may be defined differently according to a user.

Referring to FIG. 3B, due to any variable that disturbs a user's sleep, the user's sleep may change from a sleep stage corresponding to a deep sleep to a sleep stage corresponding to a light sleep (e.g., from the sleep stage N3 to the sleep stage N2, from the sleep stage N2 to the sleep stage N1, from the sleep stage N1 to the REM sleep stage, from the non-REM sleep stage to the REM sleep stage (C2), from the non-REM sleep stage to the non-sleep stage (C1), or from the REM sleep stage to the non-sleep sleep stage (C3)). For example, a sleep stage of the user may be abnormally changed by at least one of a noise value around the user, an illuminance value around the user, or a body temperature value of the user. According to an embodiment of the disclosure, a body temperature value of the user may be appropriately maintained by controlling an indoor temperature value, to prevent a sleep stage of the user from being abnormally changed, by using the control systems 200 and 250 of FIGS. 2A and 2B. According to an embodiment of the disclosure, an indoor temperature value may be controlled by using an air conditioner without opening a window or a room door, to prevent a sleep stage of the user from being abnormally changed by noise or light coming in through the opening of the window or the room door.

FIG. 4 is a conceptual diagram illustrating an operation of building a user sleep information DB by using an AI model according to an embodiment of the disclosure. For convenience of explanation, the same description as that made with reference to FIGS. 1 and 3B will be omitted.

The server device 3000 may include at least one AI model 210 and the user sleep information DB 220. The server device 3000 may analyze a correlation between a correlation between a plurality of variables (e.g., a correlation between a body temperature value of a user and a sleep stage of the user), by using the at least one AI model 210. For example, when there are a plurality of AI models 210, the plurality of AI models 210 may be trained to analyze correlations between different variables. For example, a first AI model may be trained to analyze a correlation between a body temperature value of a user and a sleep stage. For example, a second AI model may be trained to analyze a correlation between a noise value and a sleep stage. For example, a third AI model may be trained to analyze a correlation between an illuminance value and a sleep stage.

In an embodiment of the disclosure, the server device 3000 may store a correlation and data (e.g., a mapping table) corresponding to the correlation in the user sleep information DB 220. For example, the server device 3000 may generate the mapping table based on the correlation.

Referring to FIG. 4, the user sleep information DB may include a mapping table 400 corresponding to a correlation between a body temperature value and a sleep stage. For example, a body temperature range corresponding to each sleep stage may be defined in the mapping table 400. For example, in a REM sleep stage, a body temperature range upper limit may be T1 and a body temperature range lower limit may be T2. For example, in a sleep stage N1, a body temperature range upper limit may be T3 and a body temperature range lower limit may be T4. For example, in a sleep stage N2, a body temperature range upper limit may be T5 and a body temperature range lower limit may be T6.

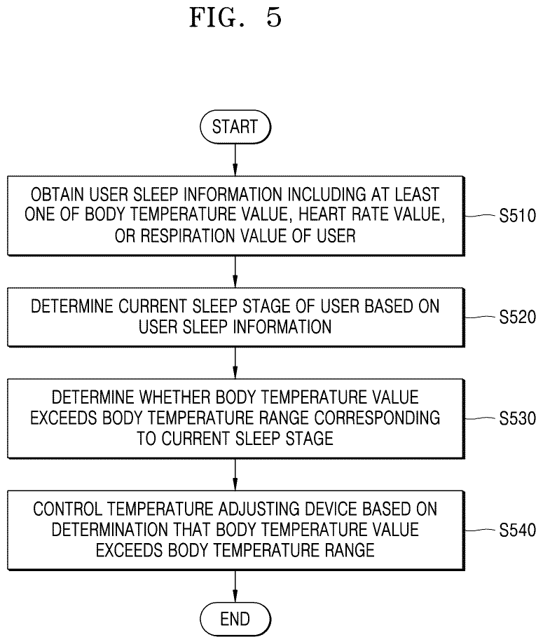

FIG. 5 is a flowchart illustrating a method of controlling a temperature adjusting device, according to an embodiment of the disclosure. The same description as that made with reference to FIGS. 1, 2A, 2B, 3A, 3B, and 4 will be omitted. For convenience of explanation, FIG. 5 will be described with reference to FIG. 2A.

Referring to FIG. 5, a method of controlling the temperature adjusting device 1000 may include operations S510 to S540. In an embodiment of the disclosure, operations S510 to S540 may be performed by the server device 3000 or a processor of the server device 3000. However, the disclosure is not limited thereto, and operations S510 to S540 may be performed by any electronic device (e.g., at least one temperature adjusting device 1000 or the wearable device 2000). A method of controlling the temperature adjusting device 1000 according to an embodiment of the disclosure is not limited to that illustrated in FIG. 5, and any one of operations illustrated in FIG. 5 may be omitted or an operation not illustrated in FIG. 5 may be further included.

In operation S510, the server device 3000 may obtain user sleep information including at least one of a body temperature value, a heart rate value, or a respiration value of a user. The user sleep information may include physiological indices indicating a sleep state or a sleep stage of the user. For example, the server device 3000 may receive user sleep information from the wearable device 2000.

In operation S520, the server device 3000 may determine a current sleep stage of the user based on the user sleep information. The server device 3000 may identify a sleep stage by analyzing at least one of the body temperature value, the heart rate value, or the respiration value of the user. For example, the server device 3000 may determine a current sleep stage of the user based on a predefined body temperature change pattern for each sleep stage of the user. For example, the server device 3000 may determine a current sleep stage of the user based on a predefined heart rate change pattern for each sleep stage of the user. For example, the server device 3000 may determine a current sleep stage of the user based on a predefined respiration change pattern for each sleep stage of the user. For example, a physiological index change pattern for each sleep stage (e.g., body temperature value, heart rate value, and respiration value) may be pre-stored in the user sleep information DB 220. In an embodiment of the disclosure, the server device 3000 may determine a current sleep stage of the user, by using an AI model using at least one of the body temperature value, the heart rate value, or the respiration value of the user as an input.

In operation S530, the server device 3000 may determine whether the body temperature value of the user exceeds a body temperature range corresponding to the current sleep stage. The body temperature range corresponding to the current sleep stage may refer to a range of body temperature values that do not deviate from a predefined sleep pattern. The predefined sleep pattern may be defined differently for each user. The predefined sleep pattern may be pre-stored in the user sleep information DB 220. For example, it is assumed that the current sleep stage of the user is a sleep stage N2. According to the predefined sleep pattern, it may be defined as not deviating from the predefined sleep pattern when the current sleep stage of the user is maintained at the sleep stage N2 or changes to a sleep stage N3. According to the predefined sleep pattern, it may be defined as deviating from the predefined sleep pattern when the current sleep stage of the user changes from the sleep stage N2 to a sleep stage N1, a REM sleep stage, or a non-sleep stage. The server device 3000 may load a correlation between a body temperature value and a sleep stage stored in the user sleep information DB 220. The server device 3000 may identify a body temperature range corresponding to the current sleep stage based on the correlation between the body temperature value and the sleep stage. The server device 3000 may determine whether a body temperature value of the user exceeds a body temperature range (e.g., a body temperature range upper limit and a body temperature range lower limit). For example, when the body temperature value of the user exceeds the body temperature range upper limit, the server device 3000 may determine that the body temperature value exceeds the body temperature range. For example, when the body temperature value of the user is less than the body temperature range lower limit, the server device 3000 may determine that the body temperature value exceeds the body temperature range. In an embodiment of the disclosure, when the body temperature value of the user is within the body temperature range, a procedure may proceed to operation S510 and the procedure up to operation S530 may be repeated.

In operation S540, the server device 3000 may control the at least one temperature adjusting device 1000 based on the determination that the body temperature value exceeds the body temperature range. The server device 3000 may increase or lower an indoor temperature value by controlling the at least one temperature adjusting device 1000. According to an embodiment of the disclosure, as the indoor temperature value is increased or lowered, the body temperature value of the user may be stably maintained within the body temperature range corresponding to the current sleep stage.

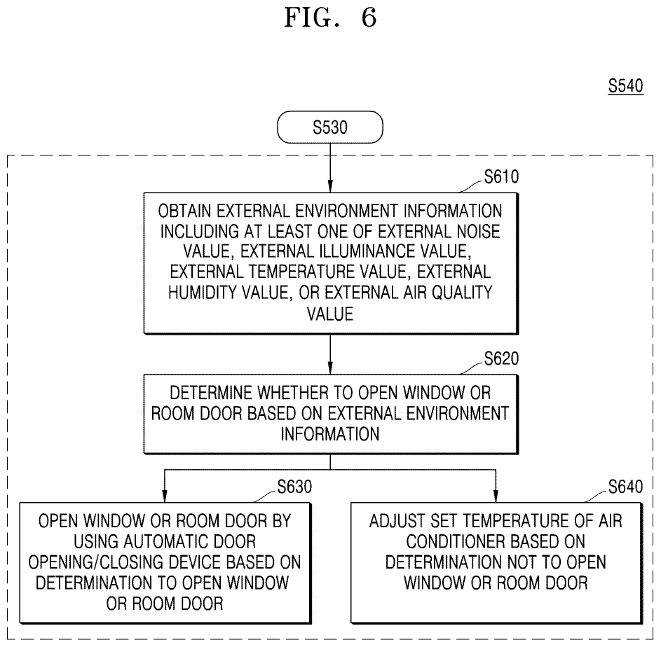

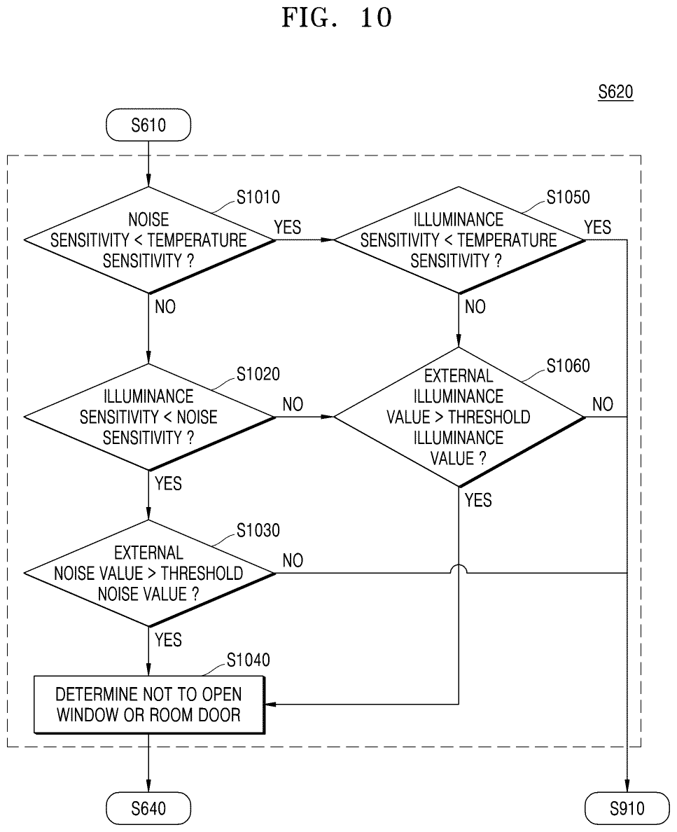

FIG. 6 is a flowchart illustrating a method of controlling a temperature adjusting device based on external environment information, according to an embodiment of the disclosure. The same description as that made with reference to FIGS. 1, 2A, 2B, 3A, 3B, 4, and 5 will be omitted. For convenience of explanation, FIG. 6 will be described with reference to FIGS. 2A and 10.

Referring to FIG. 6, operation S540 of FIG. 5 may include operations S610 to S640. In an embodiment of the disclosure, operations S610 to S640 may be performed by the server device 3000 or a processor of the server device 3000. However, the disclosure is not limited thereto, and operations S610 to S640 may be performed by any electronic device (e.g., at least one temperature adjusting device 1000 or the wearable device 2000). Detailed operations of operation S540 are not limited to those illustrated in FIG. 6, and any one of the operations illustrated in FIG. 6 may be omitted or an operation not illustrated in FIG. 6 may be further included.

In operation S610, the server device 3000 may obtain external environment information including at least one of an external noise value, an external illuminance value, an external temperature value, an external humidity value, or an external air quality value. However, the disclosure is not limited thereto, and the external environment information may include various information corresponding to the outside of an indoor space. For example, the external environment information may include various information corresponding to a space outside a room door of the indoor space (e.g., another room or a living room). For example, the external environment information may include various information corresponding to a space outside a window of the indoor space (e.g., an outdoor space). The server device 3000 may receive the external environment information from at least one external electronic device 4000. For example, the external electronic device 4000 may be any electronic device located outside the indoor space. For example, the external electronic device 4000 may include a multi-purpose sensor device that is exposed and installed in the space outside the window. Examples of the external electronic device 4000 may include a variety of home appliances located in the space outside the window (e.g., a refrigerator, an oven, a dishwasher, a washing machine, a dryer, a television (TV), a speaker, an air conditioner, a laptop computer, a desktop personal computer (PC), an air purifier, a security camera, a game console, a projector, a robot vacuum cleaner, or a smart watch).

In an embodiment of the disclosure, the external electronic device 4000 may include at least one of a microphone sensor, an illuminance sensor, a temperature sensor, a humidity sensor, or an air quality sensor. For example, the external electronic device 4000 may receive external noise by using the microphone sensor. The external electronic device 4000 may convert the received external noise into an audio signal. The external electronic device 4000 may measure an external noise value based on the audio signal. For example, the external electronic device 4000 may measure an external illuminance value by using the illuminance sensor. For example, the external electronic device 4000 may measure an external temperature value by using the temperature sensor. For example, the external electronic device 4000 may measure an external humidity value by using the humidity sensor. For example, the external electronic device 4000 may measure an external air quality value by using the air quality sensor.

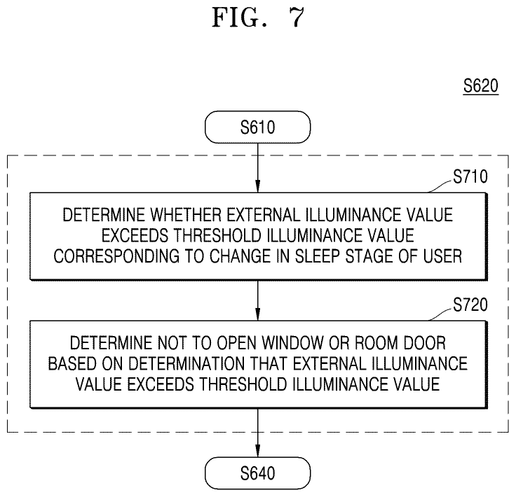

In operation S620, the server device 3000 may determine whether to open the window or the room door based on the external environment information. For example, the server device 3000 may determine whether to open the window or the room door based on at least one of the external temperature value, the external noise value, the external illuminance value, the external humidity value, or the external quality value. In an embodiment of the disclosure, the server device 3000 may compare an indoor temperature value with the external temperature value. The server device 3000 may determine whether to open the window or the room door, based on a comparison result. Detailed operations of operation S620 will be described with reference to FIGS. 7 to 11.

In operation S630, the server device 3000 may open the window or the room door by using an automatic door opening/closing device, based on the determination to open the window or the room door. For example, the server device 3000 may transmit a control signal to open the window to an automatic window opening/closing device. The automatic window opening/closing device may open the window based on the control signal. For example, the server device 3000 may transmit a control signal to open the room door to an automatic room door opening/closing device. The automatic room door opening/closing device may open the room door based on the control signal.

In operation S640, the server device 3000 may adjust a set temperature of an air conditioner, based on the determination not to open the window or the room door. In an embodiment of the disclosure, the server device 3000 may turn on the air conditioner, based on the determination not to open the window or the room door. The server device 3000 may transmit a control signal for adjusting a set temperature of the air conditioner to the air conditioner. The air conditioner may adjust a set temperature based on the control signal.

FIG. 7 is a flowchart illustrating a method of controlling a temperature adjusting device based on an external illuminance value, according to an embodiment of the disclosure. The same description as that made with reference to FIGS. 1, 2A, 2B, 3A, 3B, and 4 to 6 will be omitted. For convenience of explanation, FIG. 7 will be described with reference to FIGS. 2A and 6.

Referring to FIG. 7, operation S620 of FIG. 6 may include operations S710 and S720. In an embodiment of the disclosure, operations S710 and S720 may be performed by the server device 3000 or a processor of the server device 3000. However, the disclosure is not limited thereto, and operations S710 and S720 may be performed by any electronic device (e.g., at least one temperature adjusting device 1000 or the wearable device 2000). Detailed operations of operation S620 according to the disclosure are not limited to those illustrated in FIG. 7, and any one of the operations illustrated in FIG. 7 may be omitted or an operation not illustrated in FIG. 7 may be further included.

In operation S710, the server device 3000 may determine whether an external illuminance value exceeds a threshold illuminance value corresponding to a change in a sleep stage of a user. The threshold illuminance value may be different for each user and may be predefined. For example, when the external illuminance value exceeds the threshold illuminance value and the user is exposed to external light, a sleep stage of the user may change to a sleep stage deviating from a predefined sleep pattern. The threshold illuminance value may be pre-stored in the user sleep information DB 220. The threshold illuminance value may be pre-determined based on a correlation between an external illuminance value and a sleep stage.

In an embodiment of the disclosure, the correlation between the external illuminance value and the sleep stage may be inferred by using the AI model 210 using the external illuminance value and the sleep stage as an input. For example, the AI model 210 may be trained to infer the correlation between the external illuminance value and the sleep stage by using a dataset including an external illuminance value and a sleep stage. The server device 3000 may determine a threshold illuminance value corresponding to a sleep stage change based on the correlation between the external illuminance value and the sleep stage. In an embodiment of the disclosure, the AI model 210 may be trained to infer a threshold illuminance value for each sleep stage by using the dataset including the external illuminance value and the sleep stage. For example, the external illuminance value and the sleep stage may correspond to a specific time frame. For example, the external illuminance value and the sleep stage may be collected during the user's sleep. A method of obtaining an external illuminance value and a sleep stage has been described with reference to FIGS. 1, 2A, 2B, 3A, 3B, and 4 to 6, and thus, a repeated description thereof will be omitted.

In an embodiment of the disclosure, the server device 3000 may store the correlation between the external illuminance value and the sleep stage or data corresponding to the correlation (e.g., a mapping table) in the user sleep information DB. For example, the server device 3000 may generate a mapping table based on the correlation.

The user sleep information DB may include a mapping table corresponding to the correlation between the external illuminance value and the sleep stage. For example, a threshold illuminance value corresponding to each of sleep stages may be defined in the mapping table. For example, in a REM sleep stage, a threshold illuminance value may be a first illuminance value. For example, in a sleep stage N1, a threshold illuminance value may be a second illuminance value. For example, in a sleep stage N2, a threshold illuminance value may be a third illuminance value.

In operation S720, the server device 3000 may determine not to open a window or a room door, based on the determination that the external illuminance value exceeds the threshold illuminance value. In an embodiment of the disclosure, the external illuminance value may include a first external illuminance value indicating an illuminance value outside the window and a second external illuminance value indicating an illuminance value outside the room door. For example, the server device 3000 may determine not to open the window, based on the determination that the first external illuminance value exceeds the threshold illuminance value. For example, the server device 3000 may determine not to open the room door, based on the determination that the second external illuminance value exceeds the threshold illuminance value.

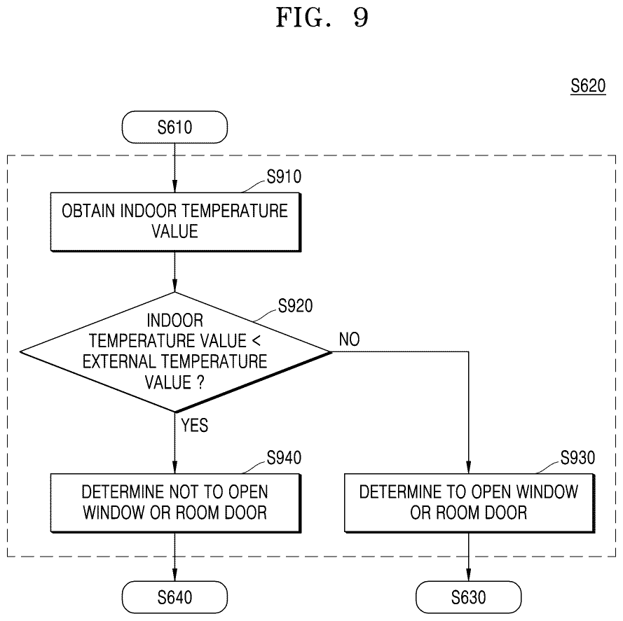

In an embodiment of the disclosure, an external temperature value may be compared with an indoor temperature value, based on the determination that the external illuminance value does not exceed the threshold illuminance value. When it is determined that the indoor temperature value may be appropriately changed by the external temperature value, the server device 3000 may determine to open the window or the room door. An operation in which the server device 3000 compares an external temperature value with an indoor temperature value will be described with reference to FIG. 9.

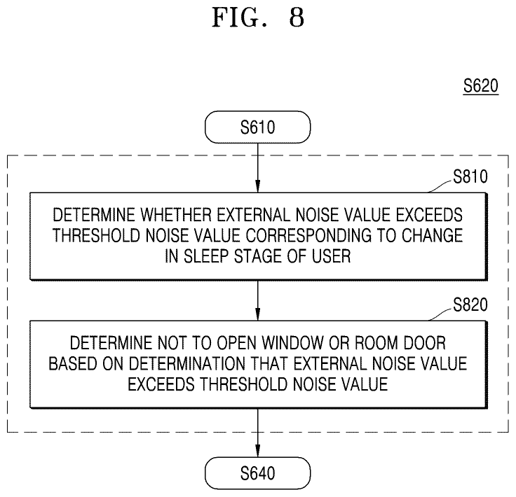

FIG. 8 is a flowchart illustrating a method of controlling a temperature adjusting device based on an external noise value, according to an embodiment of the disclosure. The same description as that made with reference to FIGS. 1, 2A, 2B, 3A, 3B, and 4 to 7 will be omitted. For convenience of explanation, FIG. 8 will be described with reference to FIGS. 2A and 6.

Referring to FIG. 8, operation S620 of FIG. 6 may include operations S810 and S820. In an embodiment of the disclosure, operations S810 and S820 may be performed by the server device 3000 or a processor of the server device 3000. However, the disclosure is not limited thereto, and operations S810 and S820 may be performed by any electronic device (e.g., at least one temperature adjusting device 1000 or the wearable device 2000). Detailed operations of operation S620 according to the disclosure are not limited to those illustrated in FIG. 8, and any one of the operations illustrated in FIG. 8 may be omitted or an operation not illustrated in FIG. 8 may be further included.

In operation S810, the server device 3000 may determine whether an external noise value exceeds a threshold noise value corresponding to a change in a sleep stage of a user. The threshold noise value may be different for each user and may be predefined. For example, when the external noise value exceeds the threshold noise value and the user is exposed to external noise, a sleep stage of the user may change to a sleep stage deviating from a predefined sleep pattern. The threshold noise value may be pre-stored in the user sleep information DB 220. The threshold noise value may be pre-determined based on a correlation between an external noise value and a sleep stage.

In an embodiment of the disclosure, the correlation between the external noise value and the sleep stage may be inferred by using the AI model 210 using the external illuminance value and the sleep stage as an input. For example, the AI model 210 may be trained to infer the correlation between the external illuminance value and the sleep stage by using a dataset including an external noise value and a sleep stage. The server device 3000 may determine a threshold noise value corresponding to a sleep stage change based on the correlation between the external noise and the sleep stage. In an embodiment of the disclosure, the AI model 210 may be trained to infer a threshold noise value for each sleep stage by using the dataset including the external noise value and the sleep stage. For example, the external noise value and the sleep stage may correspond to a specific time frame. For example, the external noise value and the sleep stage may be collected during the user's sleep. A method of obtaining an external noise value and a sleep stage has been described with reference to FIGS. 1, 2A, 2B, 3A, 3B, and 4 to 6, and thus, a repeated description thereof will be omitted.

In an embodiment of the disclosure, the server device 3000 may store the correlation between the external noise value and the sleep stage or data corresponding to the correlation (e.g., a mapping table) in the user sleep information DB. For example, the server device 3000 may generate a mapping table based on the correlation.

The user sleep information DB may include a mapping table corresponding to the correlation between the external noise value and the sleep stage. For example, a threshold noise value corresponding to each of sleep stages may be defined in the mapping table. For example, in a REM sleep stage, a threshold noise value may be a first noise value. For example, in a sleep stage N1, a threshold noise value may be a second noise value. For example, a sleep stage N2, a threshold noise value may be a third noise value.

In operation S820, the server device 3000 may determine not to open a window or a room door, based on the determination that the external noise value exceeds the threshold noise value. In an embodiment of the disclosure, the external noise value may include a first external noise value indicating an external noise value outside the window and a second external noise value indicating an external noise value outside the room door. For example, the server device 3000 may determine not to open the window, based on the determination that the first external noise value exceeds the threshold noise value. For example, the server device 3000 may determine not to open the room door, based on the determination that the second external noise value exceeds the threshold noise value.

In an embodiment of the disclosure, an external temperature value may be compared with an indoor temperature value, based on the determination that the external noise value does not exceed the threshold noise value. When it is determined that the indoor temperature value may be appropriately changed by the external temperature value, the server device 3000 may determine to open the window or the room door. An operation in which the server device 3000 compares an external temperature value with an indoor temperature value will be described with reference to FIG. 9.

FIG. 9 is a flowchart illustrating a method of controlling a temperature adjusting device by comparing an indoor temperature value with an external temperature value, according to an embodiment of the disclosure. The same description as that made with reference to FIGS. 1, 2A, 2B, 3A, 3B, and 4 to 8 will be omitted. For convenience of explanation, FIG. 9 will be described with reference to FIGS. 2A and 6.

Referring to FIG. 9, operation S620 of FIG. 6 may include operations S910 to S940. In an embodiment of the disclosure, operations S910 to S940 may be performed by the server device 3000 or a processor of the server device 3000. However, the disclosure is not limited thereto, and operations S910 to S940 may be performed by any electronic device (e.g., at least one temperature adjusting device 1000 or the wearable device 2000). Detailed operations of operation S620 according to the disclosure are not limited to those illustrated in FIG. 9, and any one of the operations illustrated in FIG. 9 may be omitted or an operation not illustrated in FIG. 9 may be further included.

In operation S910, the server device 3000 may obtain an indoor temperature value. In an embodiment of the disclosure, the server device 3000 may receive an indoor temperature value from at least one temperature adjusting device 1000, the wearable device 2000, and the external electronic device 4000. However, the disclosure is not limited thereto, and the server device 3000 may receive an indoor temperature value from any electronic device located in an indoor space and including a temperature sensor.

In operation S920, the server device 3000 may compare the indoor temperature value with an external temperature value. In FIG. 9, the following will be described assuming that the indoor temperature value should be lowered below a current value (e.g., a body temperature value exceeds a body temperature range corresponding to a current sleep stage). The server device 3000 may determine whether the indoor temperature value is lower than the external temperature value. When the indoor temperature value is lower than the external temperature value (Yes), a procedure proceeds to operation S940. When the indoor temperature value is higher than or equal to the external temperature value (No), the procedure proceeds to operation S930.

Unlike in FIG. 9, when the indoor temperature value should be increased above a current value (e.g., a body temperature is less than a body temperature range lower limit corresponding to a current sleep stage), the server device 3000 may determine whether the indoor temperature value is higher than the external temperature value. When the indoor temperature value is higher than the external temperature value, the procedure proceeds to operation S940. When the indoor temperature value is lower than or equal to the external temperature value, the procedure proceeds to operation S930.

In operation S930, the server device 3000 may determine to open a window or a room door, based on the determination that the indoor temperature value is lower than the external temperature value. Unlike in FIG. 9, when the indoor temperature value should be increased above a current value, the server device 3000 may determine to open the window or the room door, based on the determination that the indoor temperature value is higher than the external temperature value.

In operation S940, the server device 3000 may determine not to open the window or the room door, based on the determination that the indoor temperature value is higher than or equal to the external temperature value. Unlike in FIG. 9, when the indoor temperature value should be increased above a current value, the server device 3000 may determine to open the window or the room door, based on the determination that the indoor temperature value is lower than or equal to the external temperature value.

FIG. 10 is a flowchart illustrating a method of controlling a temperature adjusting device based on a noise sensitivity, an illuminance sensitivity, and a temperature sensitivity of a user, according to an embodiment of the disclosure. The same description as that made with reference to FIGS. 1, 2A, 2B, 3A, 3B, and 4 to 9 will be omitted. For convenience of explanation, FIG. 10 will be described with reference to FIGS. 2A and 6.

Referring to FIG. 10, operation S620 of FIG. 6 may include operations S1010 to S1060. In an embodiment of the disclosure, operations S1010 to S1060 may be performed by the server device 3000 or a processor of the server device 3000. However, the disclosure is not limited thereto, and operations S1010 to S1060 may be performed by any electronic device (e.g., at least one temperature adjusting device 1000 or the wearable device 2000). Detailed operations of operation S620 according to the disclosure are not limited to those illustrated in FIG. 10, and any one of the operations illustrated in FIG. 10 may be omitted or an operation not illustrated in FIG. 10 may be further included.

In operation S1010, the server device 3000 may compare a noise sensitivity with a temperature sensitivity. A noise sensitivity may indicate a magnitude of change in a sleep stage according to a change in a noise value. For example, a noise sensitivity may be an index indicating how low a threshold noise value of a user is. For example, when a noise sensitivity is high, a threshold noise value may be relatively low. For example, when a noise sensitivity is low, a threshold noise value may be relatively high. A temperature sensitivity may indicate a magnitude of change in a sleep stage according to a change in a body temperature value (e.g., an indoor temperature value). For example, a temperature sensitivity may be an index indicating how narrow a body temperature range corresponding to a sleep stage is. For example, when a temperature sensitivity is high, a body temperature range may be relatively narrow. For example, when a temperature sensitivity is low, a body temperature range is relatively wide. The server device 3000 may determine whether a noise sensitivity is lower than a temperature sensitivity. When the noise sensitivity is lower than the temperature sensitivity (Yes), a procedure proceeds to operation S1050. When the noise sensitivity is higher than or equal to the temperature sensitivity (No), the procedure proceeds to operation S1020.

In operation S1020, the server device 3000 may compare a noise sensitivity with an illuminance sensitivity. An illuminance sensitivity may indicate a magnitude of change in a sleep stage according to a change in an illuminance value. For example, an illuminance sensitivity may be an index indicating how low a threshold illuminance value of a user is. For example, when an illuminance sensitivity is high, a threshold illuminance value may be relatively low. For example, when an illuminance sensitivity is low, a threshold illuminance value may be relatively high. The server device 3000 may determine whether an illuminance sensitivity is lower than a noise sensitivity. When the illuminance sensitivity is lower than the noise sensitivity (Yes), the procedure proceeds to operation S1030. When the illuminance sensitivity is higher than or equal to the noise sensitivity (No), the procedure proceeds to operation 8910. When the procedure proceeds to operation 8910, detailed operations of operation 8620 illustrated in FIG. 9 may be performed.

In operation S1030, the server device 3000 may determine whether an external noise value exceeds a threshold noise value. Operation S1030 may correspond to operation 8810 of FIG. 8. When the external noise value exceeds the threshold noise value (Yes), the procedure proceeds to operation S1040. When the external noise value does not exceed the threshold noise value (No), the procedure proceeds to operation 8910. When the procedure proceeds to operation 8910, detailed operations of operation 8620 illustrated in FIG. 9 may be performed.

In operation 81040, the server device 3000 may determine not to open a window or a room door, based on the determination that an external illuminance value exceeds a threshold illuminance value. Operation S1040 may correspond to operation 8720 of FIG. 7 or operation 8820 of FIG. 8.

In operation S1050, the server device 3000 may compare an illuminance sensitivity with a temperature sensitivity. The server device 3000 may determine whether the illuminance sensitivity is lower than the temperature sensitivity. When the illuminance sensitivity is lower than the temperature sensitivity (Yes), the procedure proceeds to operation 8910. When the procedure proceeds to operation 8910, detailed operations of operation 8620 illustrated in FIG. 9 may be performed. When the illuminance sensitivity is higher than or equal to the temperature sensitivity (No), the procedure proceeds to operation S1060.

In operation S1060, the server device 3000 may determine whether an external illuminance value exceeds a threshold illuminance value. Operation S1030 may correspond to operation 8710 of FIG. 7. When the external illuminance value exceeds the threshold illuminance value (Yes), the procedure proceeds to operation S1040. When the external illuminance value does not exceed the threshold illuminance value (No), the procedure proceeds to operation 8910. When the procedure proceeds to operation 8910, detailed operations of operation S620 illustrated in FIG. 9 may be performed.

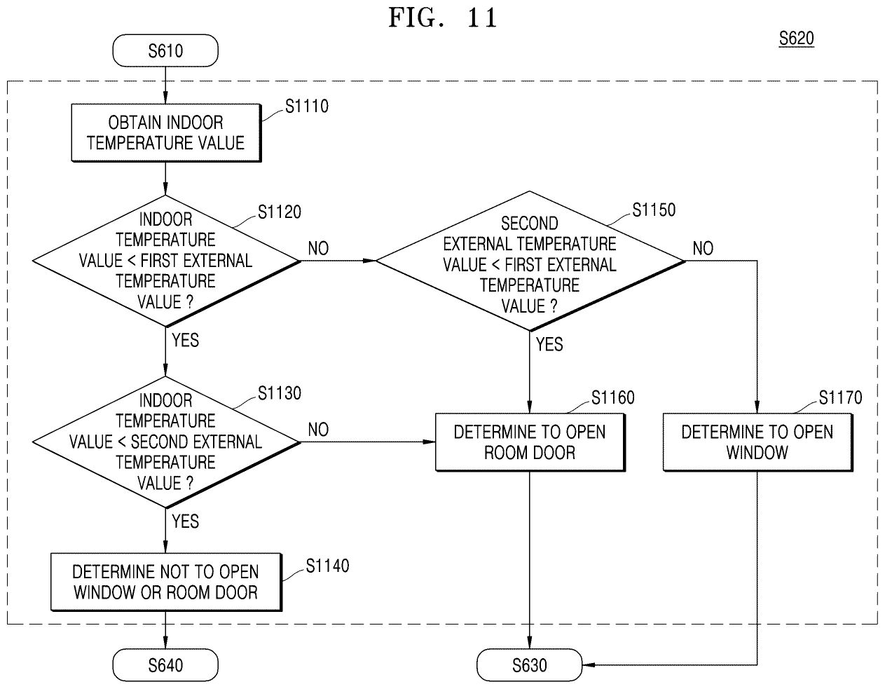

FIG. 11 is a flowchart illustrating a method of controlling a temperature adjusting device by comparing an indoor temperature value, a first external temperature value, and a second external temperature value, according to an embodiment of the disclosure. The first external temperature value may indicate a temperature value outside a window. The second external temperature value may indicate a temperature value outside a room door. The same description as that made with reference to FIGS. 1, 2A, 2B, 3A, 3B, and 4 to 10 will be omitted. For convenience of explanation, FIG. 11 will be described with reference to FIGS. 2A and 6.

Referring to FIG. 11, operation S620 of FIG. 6 may include operations S1110 to S1170. In an embodiment of the disclosure, operations S1110 to S1170 may be performed by the server device 3000 or a processor of the server device 3000. However, the disclosure is not limited thereto, and operations S1110 to S1170 may be performed by any electronic device (e.g., at least one temperature adjusting device 1000 or the wearable device 2000). Detailed operations of operation S620 according to the disclosure are not limited to those illustrated in FIG. 11, and any one of the operations illustrated in FIG. 11 may be omitted or an operation not illustrated in FIG. 11 may be further included.

In FIG. 11, the following will be described assuming that an indoor temperature value should be lowered below a current value. A case where an indoor temperature value should be increased above a current value is the same as that described with reference to FIG. 9, and thus, a detailed description thereof will be omitted.

In operation S1110, the server device 3000 may obtain an indoor temperature value. Operation S1110 is similar to operation 8910 of FIG. 9 in which the server device 3000 obtains an indoor temperature value, and thus, a detailed description thereof will be omitted.

In operation S1120, the server device 3000 may compare the indoor temperature value with a first external temperature value. The server device 3000 may determine whether the indoor temperature value is lower than the first external temperature value. When the indoor temperature value is lower than the first external temperature value (Yes), a procedure proceeds to operation S1130. When the indoor temperature value is higher than or equal to the first external temperature value (No), the procedure proceeds to operation S1150.

In operation S1130, the server device 3000 may compare the indoor temperature value with a second external temperature value. The server device 3000 may determine whether the indoor temperature value is lower than the second external temperature value. When the indoor temperature value is lower than the second external temperature value (Yes), the procedure proceeds to operation S1140. When the indoor temperature value is higher than or equal to the second external temperature value (No), the procedure proceeds to operation S1160.

In operation S1140, the server device 3000 may determine not to open a window or a room door, based on the determination that the indoor temperature value is lower than the first external temperature value and the second external temperature value.

In operation S1150, the server device 3000 may compare the first external temperature value with the second external temperature value. The server device 3000 may determine whether the second external temperature value is lower than the first external temperature value. When the second external temperature value is lower than the first external temperature value (Yes), the procedure proceeds to operation S1160. When the second external temperature value is higher than or equal to the first external temperature value (No), the procedure proceeds to operation S1170.

In operation S1160, the server device 3000 may determine to open the room door, based on the determination that the second external temperature value is lower than the first external temperature value. The server device 3000 may open the room door by controlling an automatic room door opening/closing device. According to an embodiment of the disclosure, the indoor temperature value may be lowered by introducing air outside the room door having a lower temperature value than the indoor temperature value into the room.

In operation S1170, the server device 3000 may determine to open the window, based on the determination that the second external temperature value is higher than or equal to the first external temperature value. The server device 3000 may open the window by controlling an automatic window opening/closing device. According to an embodiment of the disclosure, the indoor temperature value may be lowered, by introducing air outside the window having a lower temperature value than the indoor temperature value into the room.

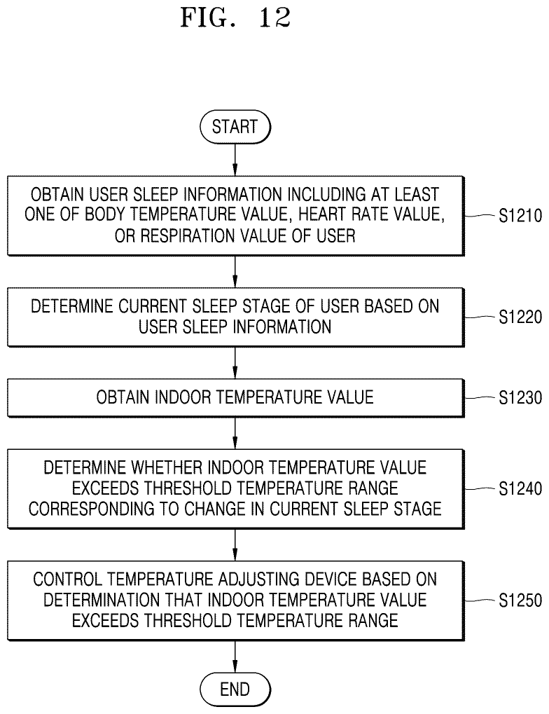

FIG. 12 is a flowchart illustrating a method of controlling a temperature adjusting device, according to an embodiment of the disclosure. The same description as that made with reference to FIGS. 1, 2A, 2B, 3A, 3B, and 4 to 11 will be omitted. For convenience of explanation, FIG. 12 will be described with reference to FIG. 2A.

Referring to FIG. 12, a method of controlling the temperature adjusting device 1000 may include operations S1210 to S1250. In an embodiment of the disclosure, operations S1210 to S1250 may be performed by the server device 3000 or a processor of the server device 3000. However, the disclosure is not limited thereto, and operations S1210 to S1250 may be performed by any electronic device (e.g., at least one temperature adjusting device 1000 or the wearable device 2000). A method of controlling the temperature adjusting device 1000 according to an embodiment of the disclosure is not limited to that illustrated in FIG. 12, and any one of operations illustrated in FIG. 12 may be omitted or an operation not illustrated in FIG. 12 may be further included. Operation S1210 corresponds to operation S510 of FIG. 5, operation S1220 corresponds to operation S520 of FIG. 5, and operation S1230 corresponds to operation 8910 of FIG. 9, and thus, a detailed description thereof will be omitted.

In operation S1240, the server device 3000 may determine whether an indoor temperature value exceeds a threshold temperature range corresponding to a current sleep stage. The threshold temperature range corresponding to the current sleep stage may refer to a range of indoor temperature values that do not deviate from a predefined sleep pattern. The server device 3000 may load a correlation between an indoor temperature value and a sleep stage stored in the user sleep information DB 220. The server device 3000 may identify a threshold temperature range corresponding to a current sleep stage based on the correlation between the indoor temperature value and the sleep stage. The server device 3000 may determine whether an indoor temperature value exceeds a threshold temperature range (e.g., a threshold temperature range upper limit and a threshold temperature range lower limit). For example, when the indoor temperature exceeds the threshold temperature range upper limit, the server device 3000 may determine that the indoor temperature exceeds the threshold temperature range. For example, when the indoor temperature value is less than the threshold temperature range lower limit, the server device 3000 may determine that the indoor temperature value exceeds the threshold temperature range. In an embodiment of the disclosure, when the indoor temperature value is within the threshold temperature range, a procedure may proceed to operation S1210 and the procedure up to operation S1240 may be repeated.

In operation S1250, the server device 3000 may control at least one temperature adjusting device 1000 based on the determination that the indoor temperature value exceeds the threshold temperature range. The server device 3000 may increase or lower an indoor temperature value by controlling the at least one temperature adjusting device 1000. According to an embodiment of the disclosure, as the indoor temperature value is increased or lowered, the indoor temperature value may be stably maintained within the threshold temperature range corresponding to the current sleep stage. For example, the server device 3000 may adjust a set temperature of an air conditioner, may open a room door by using an automatic room door opening/closing device, or may open a window by using an automatic window opening/closing device.

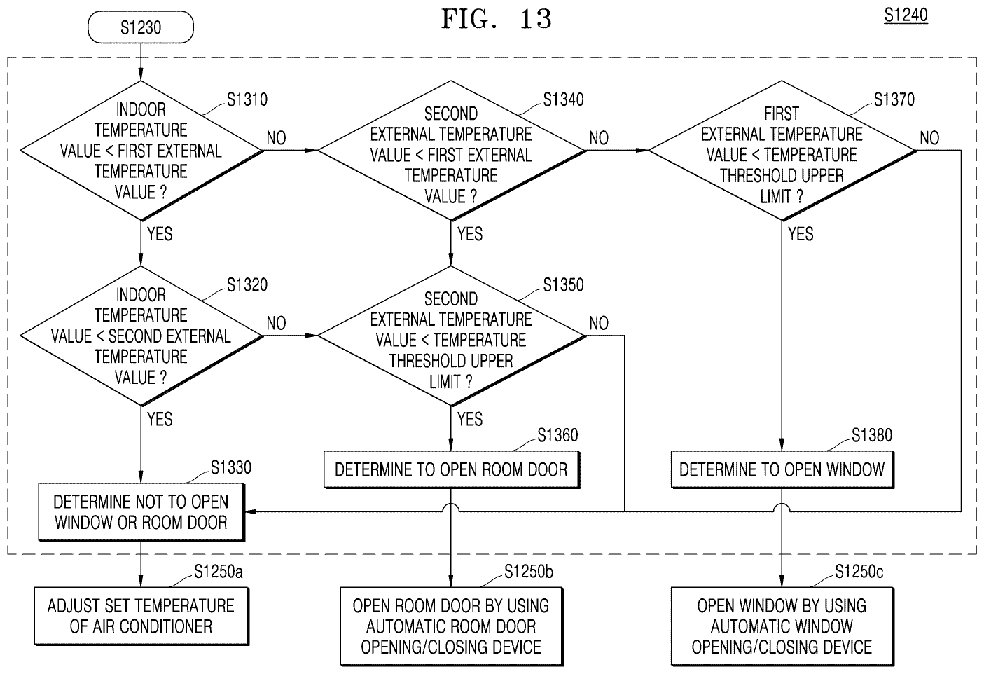

FIG. 13 is a flowchart illustrating a method of controlling a temperature adjusting device by comparing an indoor temperature value, a first external temperature value, and a second external temperature value, according to an embodiment of the disclosure. The same description as that made with reference to FIGS. 1, 2A, 2B, 3A, 3B, and 4 to 12 will be omitted. For convenience of explanation, FIG. 13 will be described with reference to FIGS. 2A and 12.

Referring to FIG. 13, operation S1240 of FIG. 12 may include operations S1310 to S1380. In an embodiment of the disclosure, operations S1310 to S1380 may be performed by the server device 3000 or a processor of the server device 3000. However, the disclosure is not limited thereto, and operations S1310 to S1380 may be performed by any electronic device (e.g., at least one temperature adjusting device 1000 or the wearable device 2000). Detailed operations of operation S1240 are not limited to those illustrated in FIG. 13, and any one of the operations illustrated in FIG. 13 may be omitted or an operation not illustrated in FIG. 13 may be further included.

In FIG. 13, the following will be described assuming that an indoor temperature value should be lowered below a current value. A case where an indoor temperature value should be increased above a current value is the same as that described with reference to FIG. 9, and thus, a detailed description thereof will be omitted.

In operation S1310, the server device 3000 may compare an indoor temperature value with a first external temperature value. The server device 3000 may determine whether the indoor temperature value is lower than the first external temperature value. When the indoor temperature value is lower than the first external temperature value (Yes), a procedure proceeds to operation S1320. When the indoor temperature value is higher than or equal to the first external temperature value (No), the procedure proceeds to operation S1340.

In operation S1320, the server device 3000 may compare an indoor temperature value with a second external temperature value. The server device 3000 may determine whether the indoor temperature value is lower than the second external temperature value. When the indoor temperature value is lower than the second external temperature value (Yes), the procedure proceeds to operation S1330. When the indoor temperature value is higher than or equal to the second external temperature value (No), the procedure proceeds to operation S1350.

In operation S1330, the server device 3000 may determine not to open a window or a room door, based on the determination that the indoor temperature value is lower than the first external temperature value and the second external temperature value. Next, the procedure proceeds to operation S1250a. In operation S1250a, the server device 3000 may adjust a set temperature of an air conditioner. According to an embodiment of the disclosure, the indoor temperature value may be lowered below a temperature threshold upper limit, by adjusting the set temperature to a temperature value lower than the indoor temperature value.

In operation S1340, the server device 3000 may compare the first external temperature value with the second external temperature value. The server device 3000 may determine whether the second external temperature value is lower than the first external temperature value. When the second external temperature value is lower than the first external temperature value (Yes), the procedure proceeds to operation S1350. When the second external temperature value is higher than or equal to the first external temperature value (No), the procedure proceeds to operation S1370.

In operation S1350, the server device 3000 may determine whether the second external temperature value is lower than the temperature threshold upper limit (e.g., also referred to as an upper limit of a threshold temperature range), based on the determination that the indoor temperature value is higher than or equal to the second external temperature value or the second external temperature value is lower than the first external temperature value. When the second external temperature value is lower than the temperature threshold upper limit (Yes), the procedure proceeds to operation S1360. When the second external temperature value is higher than or equal to the temperature threshold upper limit (No), the procedure proceeds to operation S1330.

In operation S1360, the server device 3000 may determine to open the room door, based on the determination that the second external temperature value is lower than the temperature threshold upper limit. Next, the procedure proceeds to operation S1250b. In operation S1250b, the server device 3000 may open the room door by using an automatic room door opening/closing device. According to an embodiment of the disclosure, the indoor temperature value may be lowered below the temperature threshold upper limit by introducing air outside the room door having a lower temperature value than the indoor temperature value into the room.

In operation S1370, the server device 3000 may determine whether the first external temperature value is lower than the temperature threshold upper limit, based on the determination that the second external temperature value is higher than or equal to the first external temperature value. When the first external temperature value is lower than the temperature threshold upper limit (Yes), the procedure proceeds to operation S1380. When the first external temperature value is higher than or equal to the temperature threshold upper limit (No), the procedure proceeds to operation S1330.

In operation S1380, the server device 3000 may determine to open the window, based on the determination that the first external temperature value is lower than the temperature threshold upper limit. Next, the procedure proceeds to operation S1250c. In operation S1250c, the server device 3000 may open the window by using an automatic window opening/closing device. According to an embodiment of the disclosure, the indoor temperature value may be lowered below the temperature threshold upper limit, by introducing air outside the window having a lower temperature value than the indoor temperature value into the room.

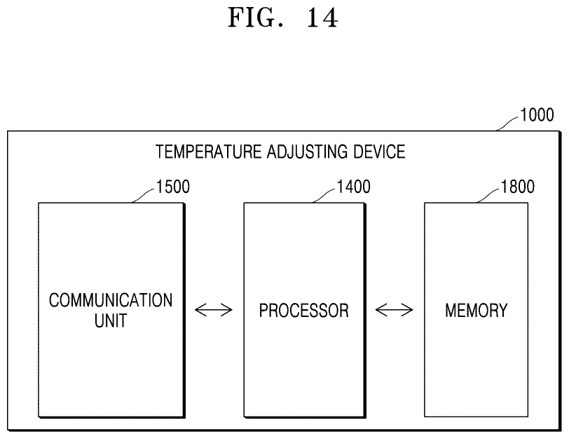

FIG. 14 is a block diagram illustrating elements of a temperature adjusting device, according to an embodiment of the disclosure. The same description as that made with reference to FIGS. 1, 2A, 2B, 3A, 3B, and 4 to 13 will be omitted.

Referring to FIG. 14, at least one temperature adjusting device 1000 according to an embodiment of the disclosure may include a processor 1400, a communication unit 1500, and memory 1800. However, not all of the illustrated elements are essential elements. The at least one temperature adjusting device 1000 may include more or fewer elements than those illustrated in FIG. 14.

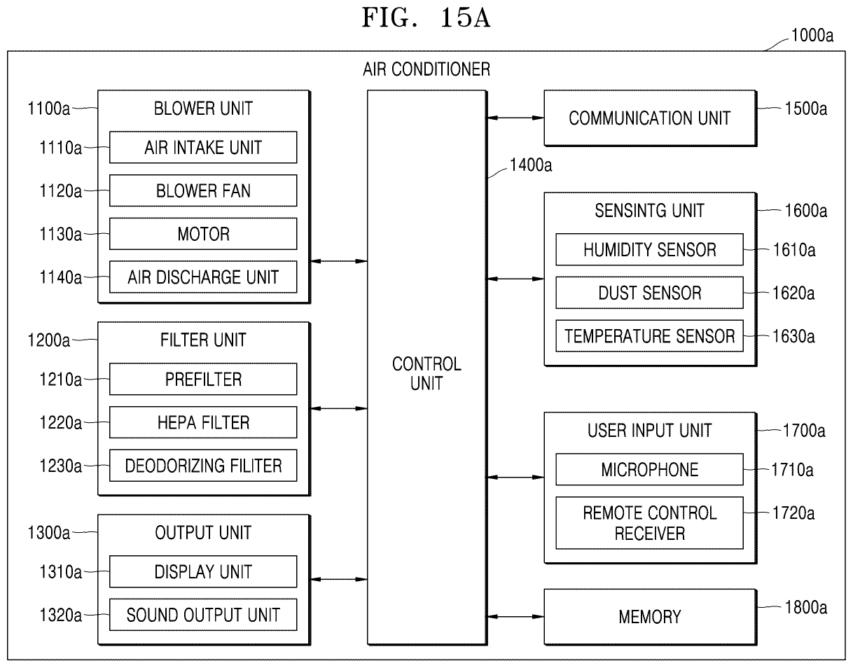

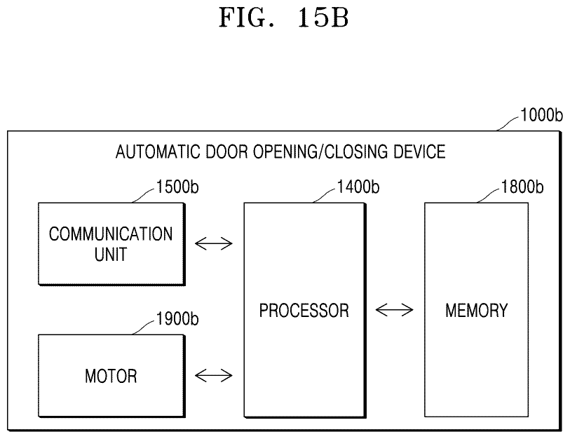

FIG. 15A is a block diagram illustrating elements of an air conditioner, according to an embodiment of the disclosure. The following will be described with reference to FIG. 14 together with FIG. 15A.

Referring to FIG. 15A, at least one temperature adjusting device 1000 according to an embodiment of the disclosure may include the air conditioner 1000a. The air conditioner 1000a may include a blower unit 1100a, a filter unit 1200a, an output unit 1300a, a processor 1400a, a communication unit 1500a, a sensing unit 1600a, a user input unit 1700a, and memory 1800a.

The blower unit 1100a may include, but is not limited to, an air intake unit 1110a, a blower fan 1120a, a motor 1130a, and an air discharge unit 1140a. The air intake unit 1110a may suck in air around the air conditioner 1000a.

The blower fan 1120a may form a flow of air so that external air is introduced into the air conditioner 1000a through the air intake unit 1110a. The blower fan 1120a may allow air purified by the filter unit 1200a to be discharged to the outside of the air conditioner 1000a through the air discharge unit 1140a. The blower fan 1120a may be rotated by the motor 1130 to form a flow of air A rotation speed (i.e., the number of rotations per minute) of the motor 1130a may be adjusted under the control of the processor 1400a.

The blower fan 1120a may include a high-pass (whirlwind) fan and a circulator fan. A plurality of high-pass fans and a plurality of circulator fans may be arranged according to an embodiment of the disclosure. The high-pass fan may emit fast and powerful cold air. For example, the high-pass fan may emit powerful cold air as soon as the high-pass fan sucks in air in a high-pass manner. The circulator fan may perform a function of sending wind away. The circulator fan may form a strong jet stream at a side wind door (circular air outlet) with the sucked wind.

The air discharge unit 1140a may include a metal cooling panel for discharging cold air and a circular air outlet. The metal cooling panel may include micro-holes for emitting cold air from sand grain-sized holes having a diameter of 1 mm. Cold air may be uniformly spread through the metal cooling panel having the micro-holes.

The filter unit 1200a may include at least one filter and may be coupled to the inside of a case body. The filter unit 1200a may be located close to the air intake unit 1110a of the air conditioner 1000a and may filter out pollutants included in air introduced from the air intake unit 1110a. According to an embodiment of the disclosure, the filter unit 1200a may include a plurality of filters. For example, the filter unit 1200a may include, but is not limited to, a prefilter 1210a, a high-efficiency particulate air (HEPA) filter 1220a, and a deodorizing filter 1230a. For example, the filter unit 1200a may further include various types of functional filters.

The prefilter 1210a may be a filter for removing relatively large particles. For example, the prefilter 1210a may remove large dust, hair, pet hair, or the like. The HEPA filter 1220a may remove fine dust, mites, viruses, and various bacteria, such as mold. The deodorizing filter 1230a may remove various indoor odors and harmful gases. The deodorizing filter 1230a may also be expressed as a carbon filter. A functional filter (not shown) may be for antibacterial purposes or to remove pollen, house dust mites, germs, bacteria, or the like. The functional filter (not shown) may include an antibacterial filter and a carbon filter.

The output unit 1300a may be used to output an audio signal or a video signal. The output unit 1300a may include a display unit 1310a and a sound output unit 1320a.

When the display unit 1310a and a touchpad form a layer structure to constitute a touchscreen, the display unit 1310a may be used as an input device in addition to an output device. The display unit 1310a may include at least one of a liquid-crystal display unit, a thin-film transistor liquid-crystal display unit, an organic light-emitting diode, a flexible display unit, a three-dimensional (3D) display unit, or an electrophoretic display unit. The air conditioner 1000a may include two or more display units 1310a according to an implementation example of the air conditioner 1000a.

In an embodiment of the disclosure, the display unit 1310a may display, but is not limited to, an operation mode, a current indoor temperature, a current indoor humidity, a current wind speed, and a set temperature (desired temperature) of the air conditioner 1000a.

The sound output unit 1320a may output audio data received from the communication unit 1500a or stored in the memory 1800a. For example, the sound output unit 1320a may output a sound signal related to a function (e.g., a notification sound or a guide voice) performed by the air conditioner 1000a.

In an embodiment of the disclosure, the output unit 1300a may include an output unit of a remote control device (remote controller). For example, an operation mode, a set temperature, a current wind speed, a current indoor temperature, a current indoor humidity, or the like, of the air conditioner 1000a may be displayed through the output unit of the remote control device (remote controller). In addition, a guide voice for a user may be output from the remote control device (remote controller).

In an embodiment of the disclosure, the output unit 1300a may further include a lighting device (not shown). For example, the output unit 1300a may indicate whether the air conditioner 1000a is operating, a pollution level of indoor air, an wind intensity, or the like, through the lighting device (not shown). The lighting device (not shown) may have various colors.

The output unit 1300a may output information related to replacement of at least one filter included in the filter unit 1200a. For example, the output unit 1300a may output text, an image (e.g., an icon), or a voice to guide a current state of a filter or the arrival of a replacement time for the filter.

The processor 1400a typically controls an overall operation of the air conditioner 1000a. For example, the processor 1400a may generally control the blower unit 1100a, the filter unit 1200a, the output unit 1300a, the communication unit 1500a, the sensing unit 1600a, the user input unit 1700a, the memory 1800a, and a power supply unit (not shown), by executing programs stored in the memory 1800a.

In an embodiment of the disclosure, the processor 1400a may include, but is not limited to, an AI processor for generating an AI model. According to an embodiment of the disclosure, the AI processor may be implemented as a chip separate from the processor 1400a. According to an embodiment of the disclosure, the AI processor may be a general-purpose chip.

In an embodiment of the disclosure, the processor 1400a may transmit current state information including at least one of operation time information of the air conditioner 1000a, set temperature information of the air conditioner 1000a, or current indoor temperature information to the server device 3000 (see FIG. 2A) through the communication unit 1500a, by executing at least one instruction stored in the memory 1800a.

The processor 1400a may receive a control signal from the server device 3000 (see FIG. 2A) through the communication unit 1500a. The processor 1400a may receive a control signal corresponding to at least one of power on, power off, set temperature increase, set temperature decrease, set windspeed increase, set wind speed decrease, set humidity increase, or set humidity decrease. For example, the processor 1400a may manage power of the air conditioner based on a control signal corresponding to power on/off. For example, the processor 1400a may adjust a set temperature based on a control signal corresponding to set temperature increase/decrease. For example, the processor 1400a may adjust a wind intensity based on a control signal corresponding to set wind speed increase/decrease. For example, the processor 1400a may adjust a set humidity based on a control signal corresponding to set humidity increase/decrease. For example, the processor 1400a may determine a sleep stage of a user based on a body temperature value of the user and a respiration value of the user. For example, the processor 1400a may train an AI model to infer a sleep stage of the user by using a body temperature value of the user and a respiration value of the user as training data. The processor 1400a may determine a sleep stage of the user, by comparing a predefined sleep pattern with the body temperature value of the user, and/or the respiration value of the user. The processor 1400a may obtain a body temperature range upper limit and a body temperature range lower limit for each sleep stage, based on the determined sleep stage. The body temperature range upper limit and the body temperature range lower limit for each sleep stage may be pre-stored in the memory 1800a. The processor 1400a may determine whether the body temperature value of the user exceeds the body temperature range upper limit or the body temperature range lower limit. The processor 1400a may perform an operation corresponding to at least one of power on, power off, set temperature increase, set temperature decrease, set wind speed increase, set wind speed decrease, set humidity increase, or set humidity decrease, based on determining whether the body temperature value of the user exceeds the body temperature range upper limit or the body temperature range lower limit. The communication unit 1500a may include one or more elements that enable communication between the air conditioner 1000a and the server device 3000 (see FIG. 2A), between the air conditioner 1000a and the wearable device 2000 (see FIG. 2A), and between the air conditioner 1000a and the external electronic device 4000 (see FIG. 2A). For example, the communication unit 1500a may include, but is not limited to, a short-range communication unit and a broadcast receiving unit.

Examples of the short-range communication unit may include, but are not limited to, a Bluetooth communication unit, a Bluetooth low energy (BLE) communication unit, a near-field communication unit, a wireless local area network (WLAN) (Wi-Fi) communication unit, a Zigbee communication unit, an infrared data association (IrDA) communication unit, a Wi-Fi direct (WFD) communication unit, an ultra-wideband (UWB) communication unit, and an Ant+ communication unit.

The broadcast receiving unit receives a broadcast signal and/or broadcast-related information from an external source through a broadcast channel. Examples of the broadcast channel may include a satellite channel and a terrestrial channel. According to an implementation example, the air conditioner 1000a may not include the broadcast receiving unit.

In an embodiment of the disclosure, the communication unit 1500a may transmit current state information to the server device 3000 (see FIG. 2A). The communication unit 1500a may transmit current state information to the server device 3000 (see FIG. 2A) at certain intervals or may transmit an indoor temperature value or current state information to the server device 3000 (see FIG. 2A) at a time requested by the server device 3000 (see FIG. 2A). The current state information may include at least one of, but not limited to, set temperature information of the air conditioner 100 or current indoor temperature information. For example, the current state information may further include set wind speed information, indoor humidity information, and device information.

The sensing unit 1600a may include, but is not limited to, a humidity sensor 1610a, a dust sensor 1620a, and a temperature sensor 1630a. For example, the sensing unit 1600a may further include a human body detection sensor, a gas sensor, a carbon dioxide sensor for measuring the concentration of carbon dioxide in air, an image sensor for detecting a location of a user, a noise measurement sensor for measuring noise, and a laser sensor for precisely detecting fine particles.

The humidity sensor 1610a may be a sensor for measuring a humidity level in air. The dust sensor 1620a may be a sensor for measuring a dust concentration in air. The temperature sensor 1630a may be a sensor for measuring a temperature of air. In an embodiment of the disclosure, the temperature sensor 1630a may be a sensor for measuring a body temperature value of the user (or an occupant) in a room. A function of each sensor may be intuitively inferred by one of ordinary skill in the art from its name, and thus, a detailed description thereof will be omitted.

The user input unit 1700a refers to a means through which the user inputs data for controlling the air conditioner 1000a. Examples of the user inputter 1700a may include, but are not limited to, a key pad, a dome switch, a touchpad (e.g., a contact capacitive type, a pressure resistive film type, an infrared (IR) sensing type, a surface ultrasonic conduction type, an integral tension measurement type, or a piezo effect type), a jog wheel, and a jog switch.

According to an embodiment of the disclosure, the user input unit 1700a may include, but is not limited to, a power button, an operation mode button (e.g., an AI comfort mode, a cooling mode, a dehumidification mode, or a cleaning mode), a wind-free function button, a temperature control button, a schedule settings button, a volume control button, a sleep button, and an automatic sterilization button.

The user input unit 1700a may further include a microphone 1710a for receiving a voice input of the user. The microphone 1710a may receive an external sound signal and may process the external sound signal into electrical sound data. For example, the microphone 1710 may receive a sound signal (e.g., a voice command) from an external device or a speaker. The microphone 1710a may use various noise removal algorithms for removing noise generated in a process of receiving an external sound signal. The microphone 1710a may receive a breathing sound of the user who is sleeping. The microphone 1710a may receive the breathing sound and may process the breathing sound into electrical sound data. The processor 1400a may obtain a respiration value based on the sound data corresponding to the breathing sound.

In an embodiment of the disclosure, the user input unit 1700a may include a remote control device (remote controller) and a remote control receiver 1720a. The remote control device (remote controller) may include, but is not limited to, a power button, a voice recognition button, an operation mode button,
a cleaning function button, a voice recognition microphone, a wind-free function button, a MAX button, a movement and control button, a temperature and air volume control button, and an additional function selection button. In an embodiment of the disclosure, when the user utters a voice command while pressing the voice recognition button of the remote control device (remote controller), the remote control device may recognize the voice command of the user.

The remote control receiver 1720a may receive a control signal from the remote control device. For example, the remote control receiver 1720a may receive a control signal input by the user from the remote control device through infrared communication.

The memory 1800a may store a program for processing and control of the processor 1400a and may store input/output data (e.g., operation mode information, user setting information, temperature data, humidity data, gas value, notification setting, filter replacement cycle information, device information, and wind speed information). The memory 1800a may store user sleep information including a predefined sleep pattern of the user.

The memory 1800a may include at least one type of storage medium from among flash memory type, a hard disk type, a multimedia card micro type, card type memory (e.g., a secure digital (SD) or extreme digital (XD) memory), random-access memory (RAM), static random-access memory (SRAM), read-only memory (ROM), electrically erasable programmable read-only memory (EEPROM), programmable read-only memory (PROM), magnetic memory, a magnetic disk, and an optical disk. Programs stored in the memory 1800a may be classified into a plurality of modules according to their functions. At least one AI model may be stored in the memory 1800a.

The air conditioner 1000a may further include a power supply unit (not shown). The power supply unit (not shown) may supply power to elements of the air conditioner 1000a under the control of the processor 1400a. The power supply unit (not shown) may supply power input from an external power source to each of the elements of the air conditioner 1000a through a power cord under the control of the processor 1400a.

FIG. 15B is a block diagram illustrating elements of an automatic door opening/closing device, according to an embodiment of the disclosure. The following will be described with reference to FIG. 14 together with FIG. 15B.

Referring to FIG. 15B, at least one temperature adjusting device 1000 according to an embodiment of the disclosure may include the automatic door opening/closing device 1000b. The automatic door opening/closing device 1000b may include a processor 1400b, a communication unit 1500b, memory 1800b, and a motor 1900b. In an embodiment of the disclosure, the automatic door opening/closing device 1000b may include at least one of an automatic window opening/closing device or an automatic room door opening/closing device.

The processor 1400b typically controls an overall operation of the automatic door opening/closing device 1000b. For example, the processor 1400b may generally control the communication unit 1500b, the memory 1800b, the motor 1900b, and a power supply unit (not shown), by executing programs stored in the memory 1800b.

In an embodiment of the disclosure, the processor 1400b may include, but is not limited to, an AI processor for generating an AI model. According to an embodiment of the disclosure, the AI processor may be implemented as a chip separate from the processor 1400b. According to an embodiment of the disclosure, the AI processor may be a general-purpose chip.

In an embodiment of the disclosure, the processor 1400b may open or close a door (e.g., a room door or a window) based on a control signal from the server device 3000 (see FIG. 2A), by executing at least one instruction stored in the memory 1800b. The processor 1400b may control the motor 1900b to automatically open or close the door.

The processor 1400b may receive a control signal from the server device 3000 (see FIG. 2A) through the communication unit 1500b. The processor 1400b may receive a control signal corresponding to at least one of door opening or door closing. For example, the processor 1400b may open or close the door based on a control signal corresponding to door opening/closing. The processor 1400b may receive a control signal corresponding to a degree to which the door is opened. The processor 1400b may determine a degree to which the door is opened based on a control signal.

The communication unit 1500b may include one or more elements that enable communication between the automatic door opening/closing device 1000b and the server device 3000 (see FIG. 2A), between the automatic door opening/closing device 1000b and the wearable device 2000 (see FIG. 2A), and between the automatic door opening/closing device 1000b and the external electronic device 4000 (see FIG. 2A). For example, the communication unit 1500b may include, but is not limited to, a short-range communication unit and a broadcast receiving unit.

Examples of the short-range communication unit may include, but are not limited to, a Bluetooth communication unit, a Bluetooth low energy (BLE) communication unit, a near-field communication unit, a wireless local area network (WLAN) (Wi-Fi) communication unit, a Zigbee communication unit, an infrared data association (IrDA) communication unit, a Wi-Fi direct (WFD) communication unit, an ultra-wideband (UWB) communication unit, and an Ant+ communication unit.

In an embodiment of the disclosure, the communication unit 1500b may receive a control signal from the server device 3000 (see FIG. 2A). The communication unit 1500b may transmit information about whether to open or close the door to the server device 3000 (see FIG. 2A).

The memory 1800b may store a program for processing and control of the processor 1400b and may store input/output data.

The memory 1800b may include at least one type of storage medium from among flash memory type, a hard disk type, a multimedia card micro type, card type memory (e.g., a secure digital (SD) or extreme digital (XD) memory), random-access memory (RAM), static random-access memory (SRAM), read-only memory (ROM), electrically erasable programmable read-only memory (EEPROM), programmable read-only memory (PROM), magnetic memory, magnetic disk, and an optical disk. Programs stored in the memory 1800b may be classified into a plurality of modules according to their functions. At least one AI model may be stored in the memory 1800b.

The automatic door opening/closing device 1000b may include the motor 1900b. The motor 1900b may rotate under the control of the processor 1400b. The door may be opened or closed through the rotation of the motor 1900b. For example, the motor 1900b may include a direct motor (DC) motor, a stepping motor, or a servo motor.

The automatic door opening/closing device 1000b may further include a power supply unit (not shown). The power supply unit (not shown) may supply power to elements of the automatic door opening/closing device 1000b under the control of the processor 1400b. The power supply unit (not shown) may supply power input from an external power source to each of the elements of the automatic door opening/closing device 1000b through a power cord under the control of the processor 1400b.

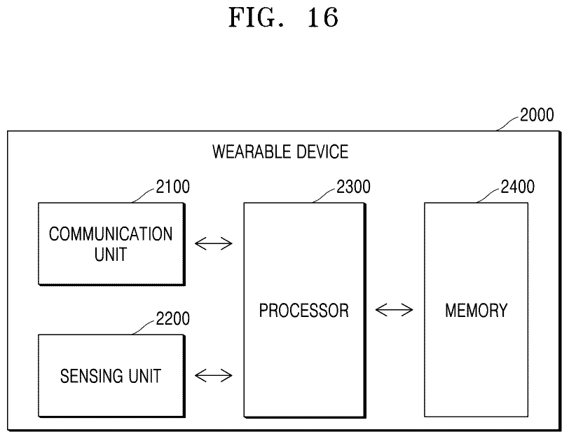

FIG. 16 is a block diagram illustrating elements of a wearable device, according to an embodiment of the disclosure. The same description as that made with reference to FIGS. 1, 2A, 2B, 3A, 3B, 4 to 14, 15A, and 15B will be omitted.

Referring to FIG. 16, the wearable device 2000 according to an embodiment of the disclosure may include a communication unit 2100, a sensing unit 2200, a processor 2300, and memory 2400. However, not all of the illustrated elements are essential elements. The wearable device 2000 may include more or fewer elements than those illustrated in FIG. 16.

Referring to FIG. 2A together with FIG. 16, the communication unit 2100 may include one or more elements that enable communication between the server device 3000 and the wearable device 2000, between at least one temperature adjusting device 1000 and the wearable device 2000, and between the external electronic device 4000 and the wearable device 2000.

In an embodiment of the disclosure, the communication unit 2100 may transmit user sleep information to the server device 3000. For example, the communication unit 2100 may transmit at least one of a body temperature value, a heart rate value, or a respiration value of a user to the server device 3000. For example, the communication unit 2100 may transmit data corresponding to a current sleep stage of the user to the server device 3000.

The sensing unit 2200 may include, but is not limited to, a body temperature measurement sensor, a respiration measurement sensor, and a heart rate measurement sensor. The body temperature measurement sensor may measure a body temperature value of the user. The respiration measurement sensor may measure a respiration value (e.g., a respiratory rate or a respiration frequency) of the user. The heart rate measurement sensor may measure a heart rate value of the user. A function of each sensor may be intuitively inferred by one of ordinary skill in the art from its name, and thus, a detailed description thereof will be omitted.

The processor 2300 typically controls an overall operation of the wearable device 2000. For example, the processor 2300 may generally control the communication unit 2100, the sensing unit 2200, the memory 2400, and a power supply unit (not shown), by executing programs stored in the memory 2400.

In an embodiment of the disclosure, the processor 2300 may include, but is not limited to, an AI processor for generating an AI model. According to an embodiment of the disclosure, the AI processor may be implemented as a chip separate from the processor 2300. According to an embodiment of the disclosure, the AI processor may be a general-purpose chip.

In an embodiment of the disclosure, the processor 2300 may process sensor data obtained by using the sensing unit 2200, by executing at least one instruction stored in the memory 2400. For example, the processor 2300 may generate user sleep information based on the sensor data. For example, the user sleep information may include a body temperature value, a respiration value, a heart rate value, and a current sleep stage.

The memory 2400 may store a program for processing and control of the processor 2300 and may store input/output data.

The memory 2400 may include at least one type of storage medium from among flash memory type, a hard disk type, a multimedia card micro type, card type memory (e.g., a secure digital (SD) or extreme digital (XD) memory), random-access memory (RAM), static random-access memory (SRAM), read-only memory (ROM), electrically erasable programmable read-only memory (EEPROM), programmable read-only memory (PROM), magnetic memory, magnetic disk, and an optical disk. Programs stored in the memory 2400 may be classified into a plurality of modules according to their functions. At least one AI model may be stored in the memory 2400.

The wearable device 2000 may further include a power supply unit (not shown). The power supply unit (not shown) may supply power to elements of the wearable device 2000 under the control of the processor 2300. The power supply unit (not shown) may supply power input from an external power source to each of elements of the wearable device 2000 through a power cord under the control of the processor 2300.

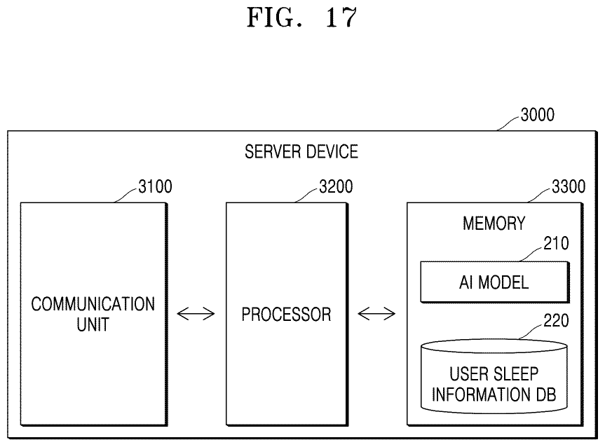

FIG. 17 is a block diagram illustrating elements of a server device according to an embodiment of the disclosure.

Referring to FIG. 2A together with FIG. 17, the server device 3000 may include a communication unit 3100, a processor 3200, and memory 3300. However, not all of the illustrated elements are essential elements. The server device 3000 may include more or fewer elements than those illustrated in FIG. 17.

The communication unit 3100 may include one or more elements that enable communication between the server device 3000 and the temperature adjusting device 1000, between the server device 3000 and the wearable device 2000, or between the server device 3000 and the external electronic device 4000.

In an embodiment of the disclosure, the communication unit 3100 may receive current state information including at least one of operation time information of the temperature adjusting device 1000, set temperature information of the temperature adjusting device 1000, or current indoor temperature information from the temperature adjusting device 1000. However, the disclosure is not limited thereto, and the communication unit 3100 may receive current humidity information, wind speed information, device information, and user setting information.

In an embodiment of the disclosure, the communication unit 3100 may receive weather information, outdoor (outside a window) humidity information, outdoor temperature information, and outdoor air quality information from an external server (e.g., a weather server). The communication unit 3100 may obtain indoor humidity information from an air purifier or a humidifier and may obtain information about whether a cooking device is used from the cooking device (e.g., a gas stove or an induction). The communication unit 3100 may obtain information about whether a lighting device that generates heat is used from a home network system. The communication unit 3100 may obtain information about whether to open/close a door (e.g., a room door or a window) from the temperature adjusting device 1000 (e.g., an automatic door opening/closing device).

The communication unit 3100 may receive location information of a user from a user terminal (e.g., a smartphone or the wearable device 2000) of the user. The communication unit 3100 may receive context information indicating a current behavior of the user (e.g., while sleeping) from the wearable device 2000 of the user. In this case, the server device 3000 may identify that the user is sleeping in a room, based on the location information of the user and the context information of the user.

The communication unit 3100 may transmit a control signal to the temperature adjusting device 1000. An operation of the temperature adjusting device 1000 corresponding to a control signal has been described above, and thus, a description thereof will be omitted.

The communication unit 3100 may provide information about the temperature adjusting device 1000 to the user through a display device (not shown). For example, the communication unit 3100 may provide information about an operation mode and the amount of power of the temperature adjusting device 1000, through a specific application executed on the display device (not shown).

The processor 3200 may control an overall operation of the server device 3000 by using a program or information stored in the memory 3300. In an embodiment of the disclosure, the processor 3200 may include an AI processor. The AI processor may be manufactured in the form of a dedicated hardware chip for AI, or may be manufactured as a part of an existing general-purpose processor (e.g., a central processing unit (CPU) or an application processor) or a dedicated graphic processor (e.g., a graphics processing unit (GPU)) and mounted on the server device 3000. The AI processor may perform an operation related to the AI model 210.

In an embodiment of the disclosure, the processor 3200 may determine a current sleep stage of the user based on user sleep information. In an embodiment of the disclosure, the user sleep information may include a current sleep stage. In an embodiment of the disclosure, the processor 3200 may determine a current sleep stage of the user corresponding to at least one of a body temperature value, a heart rate value, or a respiration value of the user, based on a predefined sleep pattern of the user stored in the user sleep information DB 220.

In an embodiment of the disclosure, the processor 3200 may determine whether the body temperature exceeds a body temperature range corresponding to the current sleep stage. In an embodiment of the disclosure, the processor 3200 may calculate a body temperature range based on a correlation between a body temperature value and a sleep stage stored in the user sleep information DB 220. In an embodiment of the disclosure, the correlation may be inferred by using the AI model 210 using a body temperature value and a sleep stage as training data. For example, the AI model 210 may be a linear regression analysis model using a body temperature value and a sleep stage as variables.

In an embodiment of the disclosure, the processor 3200 may load a body temperature range corresponding to a sleep stage stored in the user sleep information DB 220. When the body temperature value of the user is higher than a corresponding body temperature range upper limit or lower than a body temperature range lower limit, the processor 3200 may control the temperature adjusting device 1000. For example, the processor 3200 may increase or lower an indoor temperature value by controlling the temperature adjusting device 1000. The processor 3200 may generate a corresponding control signal.

The memory 3300 may store a program for processing of the processor 3200 and may store input/output data. For example, the memory 3300 may include the AI model 210 and the user sleep information DB 220.

The AI model 210 may be a model for analyzing a correlation between at least two variables. The AI model 210 may generate the user sleep information DB 220 by being trained by using the variables as training data. In an embodiment of the disclosure, the AI model 210 may update the user sleep information DB 220 as training data is added or updated and a user is added or updated.

The user sleep information DB 220 is a DB in which unique sleep information of the user is stored and may store comfortable sleep information of the user according to various conditions. For example, the user sleep information DB 220 may include, but is not limited to, a correlation between variables inferred by the AI model 210, a mapping table between variables based on the correlation, and a comfortable sleep pattern of the user.

In an embodiment of the disclosure, a method of controlling a temperature adjusting device may be provided. The method may include obtaining user sleep information including at least one of a body temperature value, a heart rate value, or a respiration value of a user. The method may include determining a current sleep stage of the user based on the user sleep information. The method may include determining whether the body temperature value exceeds a body temperature range corresponding to the current sleep stage. The method may include controlling at least one temperature adjusting device based on the determination that the body temperature value exceeds the body temperature range. According to an embodiment of the disclosure, a body temperature of the user who is sleeping may be appropriately maintained or changed so that the user experiences a comfortable sleep.

In an embodiment of the disclosure, the body temperature range may be determined by using a correlation between a body temperature value of the user and a sleep stage of the user. The correlation may be inferred by using an AI model using the body temperature value of the user and the sleep stage of the user as training data. In an embodiment of the disclosure, because the correlation is inferred through the AI model, the sleep stage of the user may be managed through an accurate correlation.

In an embodiment of the disclosure, the at least one temperature adjusting device may include at least one of an automatic door opening/closing device or an air conditioner. In an embodiment of the disclosure, a desired indoor temperature may be rapidly reached by actively adjusting an indoor temperature with the air conditioner. In an embodiment of the disclosure, power consumption of the air conditioner may be reduced by using the automatic door opening/closing device to utilize an outdoor temperature.

In an embodiment of the disclosure, the controlling of the temperature adjusting device may include obtaining external environment information including at least one of an external noise value, an external illuminance value, an external temperature value, an external humidity value, or an external air quality value. The controlling of the temperature adjusting device may include determining whether to open a window or a room door based on the external environment information. The controlling of the temperature adjusting device may include opening the window or the room door by using the automatic door opening/closing device, based on the determination to open the window or the room door. In an embodiment of the disclosure, the user may be prevented from being exposed to an outdoor environment that disturbs the user's comfortable sleep, by determining whether to open the window or the room door according to the external environment information.

In an embodiment of the disclosure, a set temperature of the air conditioner may be adjusted, based on the determination not to open the window or the room door. In an embodiment of the disclosure, the user may be prevented from being exposed to an outdoor environment that disturbs the user's comfortable sleep, by determining whether to open the window or the room door according to the external environment information.

In an embodiment of the disclosure, the determining whether to open the window or the room door based on the external environment information may include determining whether the external illuminance value exceeds a threshold illuminance value corresponding to a change in the sleep stage of the user. The determining whether to open the window or the room door based on the external environment information may include determining not to open the window or the room door, based on the determination that the external illuminance value exceeds the threshold illuminance value. In an embodiment of the disclosure, the user may be helped to experience a comfortable sleep by opening or closing the window/room door by considering an illuminance sensitivity of the user.

In an embodiment of the disclosure, the determining whether to open the window or the room door based on the external environment information may include determining whether the external noise value exceeds a threshold noise value corresponding to a change in the sleep stage of the user. The determining whether to open the window or the room door based on the external environment information may include determining not to open the window or the room door, based on the determination that the external noise value exceeds the threshold noise value. According to an embodiment of the disclosure, the user may be helped to experience a comfortable sleep by opening or closing the window/room door by considering a noise sensitivity of the user.

In an embodiment of the disclosure, the determining whether to open the window or the room door based on the external environment information may include obtaining an indoor temperature value. The determining whether to open the window or the room door based on the external environment information may include comparing the indoor temperature value with the external temperature value. The determining whether to open the window or the room door based on the external environment information may include determining whether to open the window or the room door, based on a result of the comparing.

In an embodiment of the disclosure, the comparing of the indoor temperature value with the external temperature value may include determining whether the indoor temperature value is lower than the external temperature value. The determining whether to open the window or the room door based on the result of the comparing may include determining not to open the window or the room door, based on the determination that the indoor temperature value is lower than the external temperature value. The determining whether to open the window or the room door based on the result of the comparing may include determining to open the window or the room door, based on the determination that the indoor temperature value is higher than or equal to the external temperature value.

In an embodiment of the disclosure, the external temperature value may include a first external temperature value corresponding to a temperature value outside the window and a second external temperature value corresponding to a temperature value outside the room door. The determining whether to open the window or the room door based on the external environment information may include obtaining an indoor temperature value. The determining whether to open the window or the room door based on the external environment information may include determining whether the indoor temperature value is lower than the first external temperature value. The determining whether to open the window or the room door based on the external environment information may include determining whether the indoor temperature value is lower than the second external temperature value, based on the determination that the indoor temperature value is lower than the first external temperature value. The determining whether to open the window or the room door based on the external environment information may include determining whether the second external temperature value is lower than the first external temperature value, based on the determination that the indoor temperature value is higher than or equal to the second external temperature value. The determining whether to open the window or the room door based on the external environment information may include determining not to open the window or the room door, based on the determination that the indoor temperature value is lower than the second external temperature value. The determining whether to open the window or the room door based on the external environment information may include determining to open the room door, based on the determination that the indoor temperature value is higher than or equal to the second external temperature value or the second external temperature value is lower than the first external temperature value. The determining whether to open the window or the room door based on the external environment information may include determining to open the window, based on the determination that the second external temperature value is higher than or equal to the first external temperature value. According to an embodiment of the disclosure, the user may experience of a comfortable sleep, by determining whether to open the window or the room door by considering all of an illuminance sensitivity, a noise sensitivity, and a temperature sensitivity of the user.

In an embodiment of the disclosure, the controlling of the temperature adjusting device may include obtaining an indoor temperature value. The controlling of the temperature adjusting device may include controlling the at least one temperature adjusting device based on a correlation between the indoor temperature value and the body temperature value. The user may experience a comfortable sleep, by changing an indoor temperature value by considering a change in a body temperature value of the user.

In an embodiment of the disclosure, a server device for controlling a temperature adjusting device may be provided. The server device may include memory in which at least one instruction is stored. The server device may include at least one processor configured to execute the at least one instruction. The at least one processor may be configured to obtain user sleep information including at least one of a body temperature value, a heart rate value, or a respiration value of a user. The at least one processor may be configured to determine a current sleep stage of the user based on the user sleep information. The at least one processor may be configured to determine whether the body temperature value exceeds a body temperature range corresponding to the current sleep stage. The at least one processor may be configured to control at least one temperature adjusting device based on the determination that the body temperature value exceeds the body temperature range.

A method according to an embodiment of the disclosure may be implemented as a program command executable through various computer means and may be recorded on a computer-readable medium. The computer-readable recording medium may include program commands, data files, and data structures separately or in combinations. The program commands recorded on the medium may be specially designed and configured for the disclosure or may be well-known to and be usable by one of ordinary skill in the art of computer software. Examples of the computer-readable recording medium include a magnetic medium, such as a hard disk, a floppy disk, or a magnetic tape, an optical medium, such as compact disc read-only memory (CD-ROM) or a digital versatile disc (DVD), a magneto-optical medium, such as a floptical disk, and a hardware device specially configured to store and execute program commands, such as ROM, random-access memory (RAM), or flash memory. Examples of the program commands include advanced language code that may be executed by a computer by using an interpreter or the like as well as machine language code made by a compiler.

Some embodiments of the disclosure may also be realized in the form of a recording medium including instructions executable by a computer, such as a program module executable by a computer. The computer-readable medium may be an arbitrary available medium accessible by a computer, and includes all volatile and non-volatile media and separable and non-separable media. In addition, the computer-readable medium may include a computer storage medium and a communication medium. Examples of the computer storage medium include all volatile and non-volatile media and separable and non-separable media, which have been implemented by an arbitrary method or technology, for storing information, such as computer-readable instructions, data structures, program modules, and other data. The communication medium generally includes a computer-readable instructions, a data structure, a program module, other data of a modulated data signal, such as a carrier wave, or another transmission mechanism, and an example thereof includes an arbitrary information transmission medium. Some embodiments of the disclosure may also be implemented as a computer program or a computer program product including instructions executable by a computer, such as a computer program executed by a computer.

In an embodiment of the disclosure, a machine-readable storage medium may be provided as a non-transitory storage medium. Here, ‘non-transitory’ means that the storage medium does not include a signal (e.g., an electromagnetic wave) and is tangible, but does not distinguish whether data is stored semi-permanently or temporarily in the storage medium. For example, the ‘non-transitory storage medium’ may include a buffer in which data is temporarily stored.

According to an embodiment of the disclosure, methods according to various embodiments of the disclosure may be provided in a computer program product. The computer program product is a product purchasable between a seller and a purchaser. The computer program product may be distributed in the form of a machine-readable storage medium (e.g., compact disc read-only memory (CD-ROM)), or distributed (e.g., downloaded or uploaded) online via an application store or between two user devices (e.g., smartphones) directly. When distributed online, at least part of the computer program product (e.g., a downloadable application) may be temporarily generated or at least temporarily stored in a machine-readable storage medium, such as memory of a server of a manufacturer, a server of an application store, or a relay server.

It will be appreciated that various embodiments of the disclosure according to the claims and description in the specification can be realized in the form of hardware, software or a combination of hardware and software.

Any such software may be stored in non-transitory computer readable storage media. The non-transitory computer readable storage media store one or more computer programs (software modules), the one or more computer programs include computer-executable instructions that, when executed by one or more processors of an electronic device, cause the electronic device to perform a method of the disclosure.

Any such software may be stored in the form of volatile or non-volatile storage, such as, for example, a storage device like read only memory (ROM), whether erasable or rewritable or not, or in the form of memory, such as, for example, random access memory (RAM), memory chips, device or integrated circuits or on an optically or magnetically readable medium, such as, for example, a compact disk (CD), digital versatile disc (DVD), magnetic disk or magnetic tape or the like. It will be appreciated that the storage devices and storage media are various embodiments of non-transitory machine-readable storage that are suitable for storing a computer program or computer programs comprising instructions that, when executed, implement various embodiments of the disclosure. Accordingly, various embodiments provide a program comprising code for implementing apparatus or a method as claimed in any one of the claims of this specification and a non-transitory machine-readable storage storing such a program.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. A method of controlling a temperature adjusting device, the method comprising:
obtaining user sleep information comprising at least one of a body temperature value, a heart rate value, or a respiration value of a user;
determining a current sleep stage of the user based on the user sleep information;
determining whether the body temperature value exceeds a body temperature range corresponding to the current sleep stage; and
controlling at least one temperature adjusting device based on a determination that the body temperature value exceeds the body temperature range.

2. The method of claim 1,
wherein the body temperature range is determined by using a correlation between a temperature value of the user and a sleep stage of the user, and
wherein the correlation is inferred by using an artificial intelligence model that uses the body temperature value of the user and the sleep stage of the user as training data.

3. The method of claim 2, wherein the at least one temperature adjusting device comprises at least one of an automatic door opening/closing device or an air conditioner.

4. The method of claim 3, wherein the controlling of the at least one temperature adjusting device comprises:
obtaining external environment information comprising at least one of an external noise value, an external illuminance value, an external temperature value, an external humidity value, or an external air quality value;
determining whether to open a window or a room door based on the external environment information; and
opening the window or the room door by using the automatic door opening/closing device, based on a determination to open the window or the room door.

5. The method of claim 4, further comprising adjusting a set temperature of the air conditioner, based on a determination not to open the window or the room door.

6. The method of claim 5, wherein the determining of whether to open the window or the room door based on the external environment information comprises:
determining whether the external illuminance value exceeds a threshold illuminance value corresponding to a change in the sleep stage of the user; and
determining not to open the window or the room door, based on a determination that the external illuminance value exceeds the threshold illuminance value.

7. The method of claim 6, wherein the determining of whether to open the window or the room door based on the external environment information comprises:
determining whether the external noise value exceeds a threshold noise value corresponding to a change in the sleep stage of the user; and
determining not to open the window or the room door, based on a determination that the external noise value exceeds the threshold noise value.

8. The method of claim 4, wherein the determining of whether to open the window or the room door based on the external environment information comprises:
obtaining an indoor temperature value;
comparing the indoor temperature value with the external temperature value; and
determining whether to open the window or the room door, based on a result of the comparing.

9. The method of claim 4, wherein the determining whether to open the window or the room door based on the external environment information comprises:
obtaining an indoor temperature value;
determining whether the indoor temperature value is lower than the external temperature value;
determining not to open the window or the room door, based on a determination that the indoor temperature value is lower than the external temperature value; and
determining to open the window or the room door, based on a determination that the indoor temperature value is higher than or equal to the external temperature value.

10. The method of claim 1, wherein the controlling of the at least one temperature adjusting device comprises:
obtaining an indoor temperature value; and
controlling the at least one temperature adjusting device based on a correlation between the indoor temperature value and the body temperature value.

11. A server device for controlling a temperature adjusting device, the server device comprising:
memory storing one or more computer programs; and
one or more processors communicatively coupled to the memory,
wherein the one or more computer programs include computer-executable instructions that, when executed by the one or more processors individually or collectively, cause the server device to:
obtain user sleep information comprising at least one of a body temperature value, a heart rate value, or a respiration value of a user,
determine a current sleep stage of the user based on the user sleep information,
determine whether the body temperature value exceeds a body temperature range corresponding to the current sleep stage, and
control at least one temperature adjusting device based on a determination that the body temperature value exceeds the body temperature range.

12. The server device of claim 11,
wherein the body temperature range is determined by using a correlation between a body temperature value of the user and a sleep stage of the user, and
wherein the correlation is inferred by using an artificial intelligence model that uses the body temperature value of the user and the sleep stage of the user as training data.

13. The server device of claim 12, wherein the at least one temperature adjusting device comprises at least one of an automatic door opening/closing device or an air conditioner.

14. The server device of claim 13, wherein the one or more computer programs further include computer-executable instructions that, when executed by the one or more processors individually or collectively, cause the server device to:
obtain external environment information comprising at least one of an external noise value, an external illuminance value, an external temperature value, an external humidity value, or an external air quality value;
determine whether to open a window or a room door based on the external environment information; and
open the window or the room door by using the automatic door opening/closing device, based on a determination to open the window or the room door.

15. The server device of claim 14, wherein the one or more computer programs further include computer-executable instructions that, when executed by the one or more processors individually or collectively, cause the server device to adjust a set temperature of the air conditioner, based on a determination not to open the window or the room door.

16. The server device of claim 15, wherein the one or more computer programs further include computer-executable instructions that, when executed by the one or more processors individually or collectively, cause the server device to:
determine whether the external illuminance value exceeds a threshold illuminance value corresponding to a change in the sleep stage of the user; and
determine not to open the window or the room door, based on a determination that the external illuminance value exceeds the threshold illuminance value.

17. The server device of claim 16, wherein the one or more computer programs further include computer-executable instructions that, when executed by the one or more processors individually or collectively, cause the server device to:
determine whether the external noise value exceeds a threshold noise value corresponding to a change in the sleep stage of the user; and
determine not to open the window or the room door, based on a determination that the external noise value exceeds the threshold noise value.

18. The server device of claim 14, wherein the one or more computer programs further include computer-executable instructions that, when executed by the one or more processors individually or collectively, cause the server device to:
obtain an indoor temperature value;
compare the indoor temperature value with the external temperature value; and
determine whether to open the window or the room door, based on a result of comparing the indoor temperature value with the external temperature value.

19. The server device of claim 14, wherein the one or more computer programs further include computer-executable instructions that, when executed by the one or more processors individually or collectively, cause the server device to:
obtain an indoor temperature value;
determine whether the indoor temperature value is lower than the external temperature value;
determine not to open the window or the room door, based on a determination that the indoor temperature value is lower than the external temperature value; and
determine to open the window or the room door, based on a determination that the indoor temperature value is higher than or equal to the external temperature value.

20. The server device of claim 11, wherein the one or more computer programs further include computer-executable instructions that, when executed by the one or more processors individually or collectively, cause the server device to:
obtain an indoor temperature value, and
control the at least one temperature adjusting device based on a correlation between the indoor temperature value and the body temperature value.
